(19) United States
(12) Patent Application Publication
(10) Pub. No.: US 2025/0215435 A1
(43) Pub. Date: Jul. 3, 2025

(54) ANTI-HELICASE THERAPY FOR TREATMENT OF REPEAT EXPANSION DISEASE

(71) Applicant: The Johns Hopkins University, Baltimore, MD (US)

(72) Inventors: Jiou Wang, Baltimore, MD (US); Honghe Liu, Baltimore, MD (US)

(21) Appl. No.: 18/850,645

(22) PCT Filed: Mar. 28, 2023
(86) PCT No.: PCT/US2023/065041

§ 371 Date: Sep. 25, 2024

Related U.S. Application Data
(60) Provisional application No. 63/324,846, filed on Mar. 29, 2022.

(51) Int. Cl. C12N 15/113; A61K 45/06; C07K 16/40; C12Q 1/34; C12Q 1/6883

(52) U.S. Cl. CPC C12N 15/1137; A61K 45/06; C07K 16/40; C12Q 1/34; C12Q 1/6883; C12N 2310/14; C12N 2310/531; C12Q 2600/156; C12Y 306/04012; G01N 2333/914

(57) ABSTRACT

Provided herein is technology relating to treatment of diseases e.g. neurodegenerative diseases caused by repeat-associated non-AUG translation of nucleotide repeat expansions and particularly, but not exclusively, to methods of treating disease by inhibiting DHX36 helicase by administering to the patient an inhibitor of DHX36 in order to provide an effective therapeutic intervention for treating pathogenesis related to the C9orf72 nucleotide repeat expansions.

This application claims priority to U.S. provisional patent application Ser. No. 63/324,846, filed Mar. 29, 2022, which is incorporated herein by reference in its entirety.

### STATEMENT REGARDING FEDERALLY SPONSORED RESEARCH OR DEVELOPMENT

This invention was made with government support under grant NS089616 awarded by the National Institute of Health. The government has certain rights in the invention.

### SEQUENCE LISTING

The text of the computer readable sequence listing filed herewith, titled “JHU-40257-601_SQL”, created Mar. 28, 2023, having a file size of 41,279 bytes, is hereby incorporated by reference in its entirety.

### FIELD

Provided herein is technology relating to treatment of diseases caused by repeat-associated non-AUG translation of nucleotide repeat expansions and particularly, but not exclusively, to methods of treating disease by inhibiting DHX36 helicase.

### BACKGROUND

The functions of nucleic acids are determined by their linear sequences (primary structures) and their higher-order structures (e.g., secondary and tertiary structures). G-quadruplexes are non-canonical secondary structures that form in guanine-rich nucleic acids through Hoogsteen hydrogen bonding. Putative quadruplex-forming sequences are enriched in biologically significant regions of the human genome and RNAs, including telomeres, proto-oncogene promoters, replication origins, and untranslated regions of mRNA (1, 2). G-quadruplexes are involved in diverse biological phenomena, such as gene transcription and translation, DNA replication and genome instability, telomere regulation, and immunoglobulin gene class switch recombination (3-5). Accordingly, G-quadruplexes are associated with pathogenic processes in many human diseases, including cancers and viral infections, making these unique structures potential drug targets for therapeutic intervention (6-9).

Expansions of nucleotide repeats have been linked to approximately 40 different types of genetic disorders, primarily disorders affecting neural and neuromuscular systems (10-13). For example, a hexanucleotide (GGGGCC) repeat expansion in the C9orf72 gene, comprising hundreds to thousands of repeats, is the most frequent genetic cause of both amyotrophic lateral sclerosis (ALS) and frontotemporal dementia (FTD) (14, 15). The genetics and pathologies shared by ALS, characterized by loss of motor neurons, and FTD, characterized by degeneration of the frontal and temporal lobes of the brain, suggest that these two conditions fall within the same continuous clinical spectrum (16). The proposed mechanisms of pathogenesis induced by the C9orf72 repeat expansion include loss of C9orf72 function, repeat-associated RNA toxicity, and repeat-associated non-ATG-dependent (RAN) translation (17).

RAN translation is a translational mechanism that does not require a start codon and can generate toxic repeat proteins from repeat expansions found in certain neurodegenerative disorders (18). RAN translation of the C9orf72 repeat expansion occurs in all three frames in both the sense and anti-sense transcripts, yielding five types of poly-dipeptide repeats (DPR) (17). Although several modifiers influence the production of DPR, including those involving ribosomal functions, translation initiation, and RNA processing (19-21), the mechanism of RAN translation is still poorly understood.

RNA structures play critical roles in biological processes that include translation, but the specific roles of RNA secondary structures in RAN translation remain enigmatic (22). C9orf72 GGGGCC repeat RNAs have been found to adopt G-quadruplex structures with unusually high thermal stability (23-26). However, the role of G-quadruplexes in the regulation of RAN translation and as causative agents of human disease remains unclear.

### SUMMARY

During the development of embodiments of the technology provided herein, experiments were conducted that identified a regulatory mechanism for RAN translation that is mediated by the nucleic acid structure G-quadruplex and its resolving helicase (FIG. 11). Accordingly, provided herein is technology relating to treatment of diseases caused by repeat-associated non-AUG (RAN) translation of nucleotide repeat expansions and particularly, but not exclusively, to methods of treating disease by inhibiting a helicase, e.g., DHX36 helicase.

For example, in some embodiments, the technology provides a method for treating a patient comprising a G-quadruplex ribonucleic acid (RNA). In some embodiments, methods comprise administering to the patient an inhibitor of DHX36. In some embodiments, the inhibitor of DHX36 is an antibody or antigen-binding antibody fragment. In some embodiments, the inhibitor of DHX36 is an oligonucleotide or small molecule. In some embodiments, the patient has a neurodegenerative disease. In some embodiments, the patient has ALS or FTD. In some embodiments, methods further comprise obtaining a sample from the patient and detecting a G-quadruplex RNA in the sample. In some embodiments, methods further comprise obtaining a sample from the patient after the administering and detecting a decrease in DHX36 amount or activity in the sample. In some embodiments, the patient has a hexanucleotide repeat expansion in the C9orf72 gene. In some embodiments, the patient has 10, 20, 30, 40, 50, 60, 70, 80, 90, 100, 200, 300, 400, 500, 600, 700, 800, 900, or 1000 or more hexanucleotide repeats in the C9orf72 gene.

In some embodiments, the technology provides a method for treating a patient comprising a hexanucleotide repeat expansion in the C9orf72 gene. In some embodiments, methods comprise administering to said patient an inhibitor of DHX36. In some embodiments, the inhibitor of DHX36 is an antibody or antigen-binding antibody fragment. In some embodiments, the inhibitor of DHX36 is an oligonucleotide or small molecule. In some embodiments, the patient has a neurodegenerative disease. In some embodiments, the patient has ALS or FTD. In some embodiments, methods further comprise obtaining a sample from the patient and detecting a hexanucleotide repeat expansion in the C9orf72 gene in the sample. In some embodiments, the patient has 10, 20, 30, 40, 50, 60, 70, 80, 90, 100, 200, 300, 400, 500, 600, 700, 800, 900, or 1000 or more hexanucleotide repeats in the C9orf72 gene. In some embodiments, methods further comprise obtaining a sample from the patient after the administering and detecting a decrease in DHX36 amount or activity in the sample. In some embodiments, the patient comprises a G-quadruplex RNA.

In some embodiments, the technology provides a method for treating a patient in need of treatment for a neurodegenerative disease. In some embodiments, the method comprises administering to said patient an inhibitor of DHX36. In some embodiments, the inhibitor of DHX36 is an antibody or antigen-binding antibody fragment. In some embodiments, the inhibitor of DIIX36 is an oligonucleotide or small molecule. In some embodiments, neurodegenerative disease is ALS. In some embodiments, the neurodegenerative disease is FTD. In some embodiments, methods further comprise obtaining a sample from the patient and detecting a G-quadruplex RNA in the sample. In some embodiments, methods further comprise obtaining a sample from the patient and detecting a hexanucleotide repeat expansion in the C9orf72 gene in the sample. In some embodiments, the patient has 10, 20, 30, 40, 50, 60, 70, 80, 90, 100, 200, 300, 400, 500, 600, 700, 800, 900, or 1000 or more hexanucleotide repeats in the C9orf72 gene. In some embodiments, methods further comprise obtaining a sample from the patient after the administering and detecting a decrease in DHX36 amount or activity in the sample.

Additional embodiments will be apparent to persons skilled in the relevant art based on the teachings contained herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

These and other features, aspects, and advantages of the present technology will become better understood with regard to the following drawings.

FIG. 1 is a photograph of an electrophoresis experiment showing that formation of G-quadruplexes by r(GGGGCC)4 is induced by the presence of K+. Cy3-labeled RNA probes r(GGGGCC)4, r(GTGTCC)4, and r(CCCCGG)4 were annealed for EMSA assays and resolved on a non-denaturing polyacrylamide gel in 1×TBE buffer with 75 mM KCl.

FIG. 2A to FIG. 2H show photographs of electrophoresis experiments (FIGS. 2A, 2C, 2E, and 2G) and quantification of the electrophoresis results (FIGS. 2B, 2D, 2F, and 2H) showing that DHX36 protein shows high affinity for G-quadruplex structures formed by GGGGCC RNA repeats. FIGS. 2A, 2C, 2E, and 2G show EMSA experiments performed by annealing 20 nM of four Cy5-labeled RNA probes, r(GGGGCC)8, r(GGGGCC)4, r(GTGTCC)4, and r(CCCCGG)4, gradually from 95° C. to 25° C. and then incubating them with DHX36 protein in increasing concentrations (0, 2.5, 5.0, 10, 20, 40, and 80 nM) in the presence of 100 mM KCl or LiCl. The G-quadruplexes (GQ) that specifically appeared on the gels in the presence of K+ are indicated by arrows. FIG. 2B, 2D, 2F, 2H show the results of quantification of the electrophoresis gels performed to produce binding curves of DHX36 protein to various RNA probes in the presence of K+ or Li+ (N=3), as indicated. Data are given as means t SD of three independent experiments.

FIG. 3A and FIG. 3B are photographs of electrophoresis experiments performed in duplicate showing that DHX36 shows little binding to DNA GGGGCC or CCCCGG repeats. First (FIG. 3A) and second (FIG. 2) EMSA experiments were performed by annealing 20 nM of Cy5-labeled DNA probes, d(GGGGCC)4 and d(CCCCGG)4, gradually from 95° C. to 25° C. and then incubating them with DHX36 protein in increasing concentrations (0, 2.5, 5.0, 10, 20, 40, and 80 nM) in the presence of 100 mM KCl or LiCl.

FIG. 4A to FIG. 4I show that DHX36 efficiently unwinds G-quadruplexes formed by GGGGCC RNA repeats. FIG. 4A is a schematic representation of the G-quadruplex-hemin RNAzyme system. FIG. 4B shows absorption spectra of r(GGGGCC)10, r(CCCCGG)10, and a buffer control in colorimetric reactions. [RNA]=1 μM, [Hemin]=2.5 μM, [ABTS]=2.5 μM, and [H₂O₂]=0.4 mM. In FIG. 4C, DHX36 or BSA proteins were added to the r(GGGGCC)10 RNAzyme system at increasing concentrations (0, 100, 200, 400, or 800 nM) before triggering the colorimetric reaction. Maximum absorbance at 420 nm for each group was used to compare the RNAzyme activities. Data are given as means±SD of four independent experiments. **P<0.01. FIG. 4D is a schematic of smFRET using RNA r(GGGGCC)4-U10. FIG. 4E shows FRET histograms before and after addition of the DHX36 protein. FRET values were collected from greater than 4000 molecules in 20 different fields of view. FIG. 4F shows two sets of representative smFRET traces upon addition of DHX36 (arrows). The smFRET trace shows fluorescence intensities observed for Cy3 (green) and Cy5 (red) and the calculated FRET efficiency (blue) when 10 nM DHX36 protein was added. FIG. 4G shows the smFRET trace taken after DHX36 was added to the G4C2-U10 substrate. FIG. 4H shows FRET histograms taken 3, 10, and 20 minutes after 1 mM ATP was added to the DHX36 and G4C2-U10 substrate mix. FIG. 4I shows three sets of representative smFRET traces after ATP was added.

FIG. 5A to FIG. 5E show that DHX36 binding to C9orf72 repeat RNA promotes efficient RAN translation in cells. FIG. 5A is a schematic of the inducible luciferase-based C9orf72 RAN translation reporter system in HeLa Flp-In cells. The wide and narrow rectangles represent exons and introns, respectively. FIG. 5B shows fluorescence microscope images of RNA FISII with a (CCCCGG)4-Cy3 probe demonstrated repeat RNA foci in the cells expressing the (GGGGCC)70. FIG. 5C shows DHX36 knockdown significantly decreased C9orf72 RAN translation efficiency, measured as the ratio of Nano luciferase signal to firefly luciferase signal, in the HeLa Flp-In cells (left, N=3). The knockdown of DHX36 was confirmed by immunoblot analysis of the cells expressing (GGGGCC)₇₀ (right). FIG. 5D shows the results of RNA immunoprecipitation experiments in HeLa Flp-In cells with or without (GGGGCC)₇₀ showing the enrichment of (GGGGCC)₇₀-containing transcripts (Nano Luc RNAs) but not the internal control firefly Luc transcripts upon pulldown of DHX36 (N=3). FIG. 5E shows that immunoblotting confirmed the pulldown of DHX36 in the RNA immunoprecipitation experiments in HeLa Flp-In cells with and without (GGGGCC)70. Data are given as means t SD of three independent experiments. *P<0.05, **P<0.01.

FIG. 6A to FIG. 6D show that DHX36 promotes efficient translation elongation through GGGGCC repeat RNAs. FIG. 6A is a schematic of the reporter constructs used to monitor the effects of (GGGGCC)n repeats on translation elongation. In FIG. 6B, the effects of (GGGGCC)n repeats on transcription were compared by RT-PCR analyses of EGFP RNA levels in HEK293 cells expressing reporter constructs containing various GGGGCC repeats and non-G-quadruplex-forming control sequences (non-GQ-mers) of the same sizes. The level of EGFP RNA was normalized to that of NeoR RNA, which is independently expressed on the same reporter construct. FIG. 6C shows immunoblot analysis of EGFP from HEK293 cells expressing the reporter and control constructs. β-actin was used as the loading control.

FIG. 6D shows immunoblot analysis of EGFP from HEK293 cells expressing the reporter and control constructs after the cells were treated with shRNAs against DHX36 or non-targeting control shRNAs. Data are given as means±SD of three independent experiments. *P<0.05, **P<0.01.

FIG. 7A to FIG. 7C show reduction in poly-GP by knockdown of DHX36 in patient-derived iPSCs and iMNs and upregulation of DHX36 in the spinal cords of C9orf72-linked ALS patients. In FIG. 7A, C9orf72-linked ALS patient-derived iPSCs were analyzed for poly-GP levels by ELISA, with or without the stable knockdown of DHX36. Data are given as means t SD of four replicates from two independent experiments. **P<0.01, ***P<0.001. In FIG. 7B, C9orf72-linked ALS patient-derived iMNs were analyzed for poly-GP levels by ELISA, with or without the stable knockdown of DHX36. Data are given as means±SD of four replicates from two independent experiments. ***P<0.001, ****P<0.0001. FIG. 7C shows that DHX36 protein expression levels are higher in the spinal cord tissues from C9orf72-linked ALS patients than in the controls. Data are given as means±SD of samples from five patients or five controls. *P=0.0335.

FIG. 8A to FIG. 8C show that GGGGCC repeat RNAs show G-quadruplex-hemin RNAzyme activity that is negatively regulated by DHX36. FIG. 8A shows absorption spectra of r(GGGGCC)4, r(GGGGCC)8, r(GGGGCC)10, and the buffer control in the colorimetric reactions. FIG. 8B shows the results of colorimetric reaction experiments in which DHX36 or BSA proteins were added to the r(GGGGCC)8 RNAzyme system at increasing concentrations (0, 400, or 800 nM). FIG. 8C shows the results of colorimetric reaction experiments in which DHX36 or BSA proteins were added to the r(GGGGCC)4 RNAzyme system at increasing concentrations (0, 400, or 800 nM).

FIG. 9A and FIG. 9B show that GGGGCC repeat RNAs in the open reading frame impair in vitro translation. FIG. 9A is a schematic of the constructs for the in vitro translation system. FIG. 9B show results from real-time monitoring of translation using wheat germ extract. EGFP fluorescence value was recorded every two minutes.

FIG. 10 shows the results of quantifying the EGFP mRNA levels in FIG. 6D by qPCR. The level of EGFP mRNA was normalized to that of the internal control gene ACTB. Data are given as means t SD from three independent experiments.

FIG. 11 is a schematic showing a model for DHX36-mediated C9orf72 RAN translation regulation. C9orf72 repeat RNA forms G-quadruplex structures that repress RAN translation. DHX36 efficiently unwinds the G-quadruplex structure to facilitate RAN translation, resulting in the production of dipeptide repeat proteins.

FIG. 12 shows a generic structure of a small molecule that stabilizes r(GGGGCC)n quadruplexes (59). The molecule comprises two linked five-membered rings each comprising a group 16 atom (e.g., where X=O, S, Se, etc.)

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to treatment of diseases caused by repeat-associated non-AUG translation of nucleotide repeat expansions and particularly, but not exclusively, to methods of treating disease by inhibiting DHX36 helicase.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the terms “about”, “approximately”, “substantially”, and “significantly” are understood by persons of ordinary skill in the art and will vary to some extent on the context in which they are used. If there are uses of these terms that are not clear to persons of ordinary skill in the art given the context in which they are used, “about” and “approximately” mean plus or minus less than or equal to 10% of the particular term and “substantially” and “significantly” mean plus or minus greater than 10% of the particular term.

As used herein, disclosure of ranges includes disclosure of all values and further divided ranges within the entire range, including endpoints and sub-ranges given for the ranges. As used herein, the disclosure of numeric ranges includes the endpoints and each intervening number therebetween with the same degree of precision. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

As used herein, the suffix “-free” refers to an embodiment of the technology that omits the feature of the base root of the word to which “-free” is appended. That is, the term “X-free” as used herein means “without X”, where X is a feature of the technology omitted in the “X-free” technology. For example, a “calcium-free” composition does not comprise calcium, a “mixing-free” method does not comprise a mixing step, etc.

Although the terms “first”, “second”, “third”, etc. may be used herein to describe various steps, elements, compositions, components, regions, layers, and/or sections, these steps, elements, compositions, components, regions, layers, and/or sections should not be limited by these terms, unless otherwise indicated. These terms are used to distinguish one step, element, composition, component, region, layer, and/or section from another step, element, composition, component, region, layer, and/or section. Terms such as “first”, “second”, and other numerical terms when used herein do not imply a sequence or order unless clearly indicated by the context. Thus, a first step, element, composition, component, region, layer, or section discussed herein could be termed a second step, element, composition, component, region, layer, or section without departing from technology.

As used herein, an “increase” or a “decrease” refers to a detectable (e.g., measured) positive or negative change, respectively, in the value of a variable relative to a previously measured value of the variable, relative to a pre-established value, and/or relative to a value of a standard control. An increase is a positive change preferably at least 10%, more preferably 50%, still more preferably 2-fold, even more preferably at least 5-fold, and most preferably at least 10-fold relative to the previously measured value of the variable, the pre-established value, and/or the value of a standard control. Similarly, a decrease is a negative change preferably at least 10%, more preferably 50%, still more preferably at least 80%, and most preferably at least 90% of the previously measured value of the variable, the pre-established value, and/or the value of a standard control. Other terms indicating quantitative changes or differences, such as “more” or “less,” are used herein in the same fashion as described above.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject. In some embodiments, the subject has a neurodegenerative disorder (e.g., amyotrophic lateral sclerosis, frontotemporal dementia).

As used herein, the term “non-human animals” refers to all non-human animals including, but not limited to, vertebrates such as rodents, non-human primates, ovines, bovines, ruminants, lagomorphs, porcines, caprines, equines, canines, felines, aves, etc.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used herein, the term “in vitro” refers to an artificial environment and to processes or reactions that occur within an artificial environment. In vitro environments can comprise, but are not limited to, test tubes and cell culture. The term “in vivo” refers to the natural environment (e.g., an animal or a cell) and to processes or reaction that occur within a natural environment.

As used herein, the terms “test compound” and “candidate compound” refer to any chemical entity, pharmaceutical, drug, and the like that is a candidate for use to treat or prevent a disease, illness, sickness, or disorder of bodily function (e.g., a neurodegenerative disorder (e.g., amyotrophic lateral sclerosis, frontotemporal dementia)). Test compounds comprise both known and potential therapeutic compounds. A test compound can be determined to be therapeutic by screening using the screening methods of the present disclosure.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, and industrial samples. Such examples are not however to be construed as limiting the sample types applicable to the present disclosure.

As used herein, the term “effective amount” refers to the amount of a compound (e.g., a compound described herein) sufficient to effect beneficial or desired results. An effective amount can be administered in one or more administrations, applications or dosages and is not limited to or intended to be limited to a particular formulation or administration route.

As used herein, the term “co-administration” refers to the administration of at least two agent(s) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

As used herein, the term “pharmaceutical composition” refers to the combination of an active agent with a carrier, inert or active, making the composition especially suitable for diagnostic or therapeutic use in vivo or ex vivo.

As used herein, the term “antigen-binding agent” (e.g., “antigen-binding protein” or protein mimetic such as an apatamer) refers to proteins that bind to a specific antigen. “Antigen-binding proteins” include, but are not limited to, immunoglobulins, including polyclonal, monoclonal, chimeric, single chain, single domain, scFv, minibody, nanobody, and humanized antibodies, Fab fragments, F(ab′)2 fragments, and Fab expression libraries.

As used herein, the term “single-chain variable fragment” (scFv) refers to an antibody fragment that comprises a fusion protein of the variable regions of the heavy (VH) and light chains (VL) of an immunoglobulin. In some embodiments, the VH and VL are connected with a short linker peptide.

As used herein, the term “minibody” refers to an antibody fragment that retains antigen binding activity. In some embodiments, minibodies comprise an scFv fused to an Fe region (e.g., an IgG Fc region).

Various procedures known in the art are used for the production of polyclonal antibodies. For the production of antibody, various host animals can be immunized by injection with the peptide or protein containing the desired epitope including but not limited to rabbits, mice, rats, sheep, goats, llamas, alpacas, camels, etc. In a preferred embodiment, the peptide is conjugated to an immunogenic carrier (e.g., diphtheria toxoid, bovine serum albumin (BSA), or keyhole limpet hemocyanin (KLH)). Various adjuvants are used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, Gerbu adjuvant and potentially useful human adjuvants such as BCG (Bacille Calmette-Guerin) and Corynebacterium parvum.

For preparation of monoclonal antibodies, any technique that provides for the production of antibody molecules by continuous cell lines in culture may be used (See, e.g., Harlow and Lane, Antibodies: A Laboratory Manual, Cold Spring Harbor Laboratory Press, Cold Spring Harbor, NY). These include, but are not limited to, the hybridoma technique originally developed by Köhler and Milstein (Köhler and Milstein (1975) Nature, 256: 495-497) and the trioma technique, the human B-cell hybridoma technique (See e.g., Kozbor (1983) Immunol. Today, 4: 72), and the EBV-hybridoma technique to produce human monoclonal antibodies (Cole (1985) in Monoclonal Antibodies and Cancer Therapy, Alan R. Liss, Inc., pp. 77-96). In some embodiments, suitable monoclonal antibodies, including recombinant chimeric monoclonal antibodies and chimeric monoclonal antibody fusion proteins, are prepared as described herein.

According to the technology, techniques described for the production of single chain antibodies (see, e.g., U.S. Pat. No. 4,946,778; herein incorporated by reference) can be adapted to produce specific single chain antibodies as desired. An additional embodiment of the technology utilizes the techniques known in the art for the construction of Fab expression libraries (e.g., Huse (1989) Science, 246: 1275-1281) to allow rapid and easy identification of monoclonal Fab fragments with the desired specificity.

In some embodiments, monoclonal antibodies are generated using the ABL-MYC method (see, e.g., U.S. Pat. Nos. 5,705,150 and 5,244,656, each of which is herein incorporated by reference). ABL-MYC is a recombinant retrovirus that constitutively expresses v-abl and c-myc oncogenes. When used to infect antigen-activated splenocytes, this retroviral system rapidly induces antigen-specific plasmacytomas. ABL-MYC targets antigen-stimulated (Ag-stimulated) B-cells for transformation.

In some embodiments, biopanning (see, e.g., Pardon (2014) Nat Protoc. 9(3): 674-93) is used to generate single domain antibodies. In some embodiments, to generate murine scFv units, phage-based biopanning strategies, of which there are several published protocols available, are used.

Antibody fragments that contain the idiotype (antigen binding region) of the antibody molecule can be generated by known techniques. For example, such fragments include but are not limited to: the F(ab′)2 fragment that can be produced by pepsin digestion of an antibody molecule; the Fab′ fragments that can be generated by reducing the disulfide bridges of an F(ab′)2 fragment, and the Fab fragments that can be generated by treating an antibody molecule with papain and a reducing agent.

Genes encoding antigen-binding proteins can be isolated by methods known in the art. In the production of antibodies, screening for the desired antibody can be accomplished by techniques known in the art (e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays, etc.), complement fixation assays, immunofluorescence assays, protein A assays, phage display biopanning, and immunoelectrophoresis assays, etc.)

As used herein, the term “toxic” refers to any detrimental or harmful effects on a cell or tissue as compared to the same cell or tissue prior to the administration of the toxicant.

### DESCRIPTION

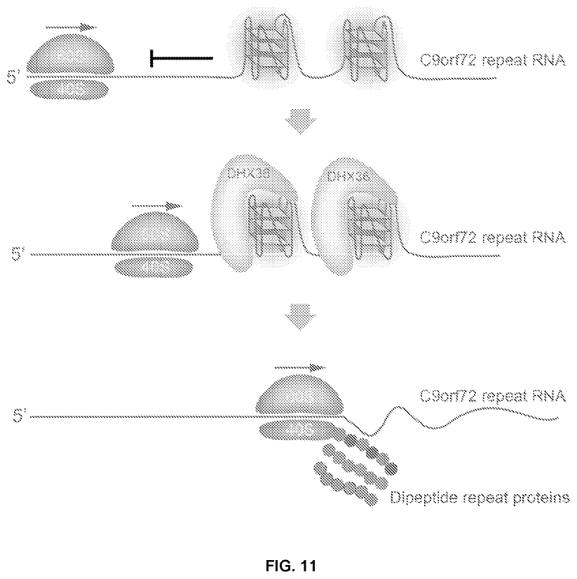

During the development of embodiments of the technology described herein, experiments were conducted to identify a regulatory mechanism for non-canonical (e.g., RAN) translation. Data collected from experiments indicated that a helicase (e.g., DHX36 helicase) regulates non-canonical (e.g., RAN) translation. In particular, the data indicated that DHX36 regulates RAN translation that occurs at repeat expansions (e.g., comprising GGGGCC repeats (e.g., as found at and transcribed from C9orf72)) that form stable G-quadruplexes (see, e.g., FIG. 11).

### G-Quadruplexes

G-quadruplex sequence motifs are frequently found throughout the genome. G-quadruplex sequences form helical secondary structures of 1, 2, or 4 strands that are stabilized by guanine tetrads. In particular, the four guanine bases of the quadruplex associate through Hoogsteen hydrogen bonding to form a square planar structure called a guanine tetrad (G-tetrad or G-quartet), and two or more guanine tetrads stack on top of each other to form a G-quadruplex. An exemplary G-quadruplex sequence is GGGGCC. When present in RNA, the GGGGCC repeat sequences form parallel G-quadruplex structures; when present in DNA, GGGGCC repeat sequences form anti-parallel G-quadruplexes (23, 25-26, 35).

G-quadruplex sequence motifs are often found near telomeres and in non-coding regions, e.g., in transcriptional regulatory regions (e.g., promotors) of genes in microbes and eukaryotes. In humans, G-quadruplex sequences have been found near oncogenes and other disease-causing or disease-associated genetic loci. G-quadruplexes are rarely found in coding sequences, where stable higher-order structures would impede ribosome scanning (54) and thus reduce translation efficiency.

While GGGGCC repeats reduce translation efficiency, translation has been observed to progress through the G-quadruplex structure to produce DPR in cells. These observations indicate that cellular machinery exists to unwind G-quadruplexes or lock them in unfolded states to allow for ribosome scanning along transcripts. Data collected during the development of embodiments of the technology described herein indicated that knockdown of DHX36 suppressed translation elongation at the GGGGCC repeats and decreased the production of poly-GP. Accordingly, DHX36 is actively engaged in resolving stable G-quadruplexes and thus enhances ribosome processivity.

### RNA Helicases

RNA helicases constitute a large family of RNA structure-modulating proteins that are involved in many aspects of RNA metabolism (46). In particular, DHX36 is a member of the DEAH-Box helicase family. DHX36 binds to RNA G-quadruplexes (e.g., with high affinity) and is a major source of resolving RNA quadruplexes in HeLa cell lysates (47). Moreover, DHX36 binds to RNA G-quadruplexes in living bacterial cells (48). Accordingly, DHX36 generally binds to G-quadruplexes produced by organisms across eukaryotic and bacterial taxa. Further, RNA G-quadruplexes are globally unfolded in eukaryotic cells (49), indicating that endogenous RNA helicase activities in eukaryotes (e.g., DHX36) resolve these structures. In addition, DHX36 promotes the effective translation of G-quadruplex-containing mRNAs (50-53).

The in vivo activities of DHX36 on C9orf72 repeat-associated translation accord with in vitro (e.g., biochemical and biophysical) analyses indicating that DHX36 recognizes and resolves r(GGGGCC)n repeats. DHX36 binds to GGGGCC repeat RNA G-quadruplexes with specificity and high affinity, and DHX36 resolves GGGGCC repeat RNA G-quadruplexes in an ATP-dependent repetitive un-folding and refolding maneuver. DHX36 preferentially binds to the parallel form of G-quadruplexes (36) found in RNA relative to the anti-parallel form of G-quadruplexes formed in DNA. Structural studies have produced a co-crystal structure of DHX36 bound to a parallel DNA G-quadruplex, which showed close contacts between DHX36 and the quadruplex and showed additional contacts of DHX36 with an overhanging single-stranded DNA. Studies of the mechanism of resolving the G-quadruplex structure in DNA indicated that DHX36 binds to the G-quadruplex substrate, and DHX36 resolves the DNA quadruplex structure by repetitively pulling a single guanine base out of the folded quadruplex structure (27, 42). Importantly, a similar repetitive unfolding activity resolves RNA G-quadruplexes (43). Thus, the DHX36 protein resolves G-quadruplexes on GGGGCC repeat RNAs, e.g., by binding to the RNA with the DHX36 binding domain and actively and repetitively unfolding the G-quadruplex with the DHX36 catalytic domains. As a consequence, the DHX36 helicase participates in dynamic regulation of translation that promotes RAN translation.

Nucleotide sequences for DHX36 messenger RNA transcripts are provided by SEQ ID NO: 25 (transcript variant 1) and by SEQ ID NO: 26 (transcript variant 2). Amino acid sequences for the DHX36 polypeptide isoforms are provided by SEQ ID NO: 27 (isoform 1) and by SEQ ID NO: 28 (isoform 2).

### G-Quadruplexes and DHX36 in Disease

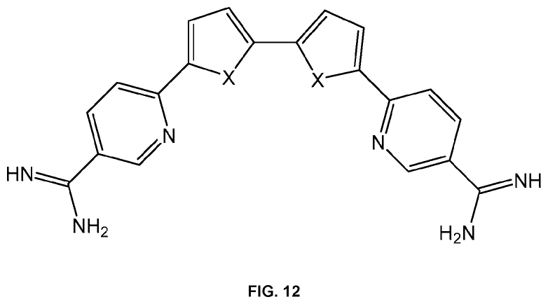

Quadruplex nucleic acids have been targeted by treatments for cancer, viral infections, and for developmental disorders (e.g., ATR-X syndrome) (55-58). Further, a group of small molecules that stabilize r(GGGGCC)n quadruplexes reduced DPR products in fly and mammalian cell models (59). These molecules (e.g., DB1246, DB1247, and DB1273) comprise two linked five-membered rings each comprising a group 16 atom (e.g., 0, S, Se, etc.) (FIG. 12).

In addition, other small molecules that bind a hairpin form of the r(GGGGCC)n repeat and a helicase (e.g., DDX3X) that recognizes the same hairpin structure have both been found to negatively regulate DPR production (21, 60). Accordingly, while hairpins promote RAN translation, G-quadruplexes obstruct RAN translation occurring at GGGGCC repeats.

During the development of embodiments of the technology described herein, data were collected that identified helicase activity (e.g., by DHX360 as promoting RAN translation. In particular, helicases (e.g., DHX36) resolve G-quadruplex secondary structures and/or promote conformational transition of G-quadruplexes that decrease and/or minimize translation at GGGGCC repeats to hairpins that increase and/or maximize translation at GGGGCC repeats.

During the development of embodiments of the technology described herein, data were collected that showed abnormal accumulation of DHX36 protein in spinal cord tissues of C9orf72-linked ALS patients. These data indicated that the upregulation of DHX36 contributes to RAN translation and thus also causes the associated pathologies of patients having C9orf72 repeat expansions. Further, G-quadruplexes present within C9orf72 repeat RNAs are involved in other cellular processes such as nucleocytoplasmic transport, alternative splicing, phase separation, and assembly of membraneless organelles such as paraspeckles (61-64). Thus, DHX36 is also predicted to be associated with pathologies involving nucleocytoplasmic transport, alternative splicing, phase separation, and assembly of membraneless organelles. In addition, DHX36 protein prevents the accumulation of translationally inactive mRNAs with G-quadruplexes and is associated with, and affects, the formation of stress granules (50, 65), which are stress-induced organelles implicated in ALS/FTD pathogenesis (66).

Thus, the role of DHX36 in RAN translation indicates that targeting helicase activity (e.g., DHX36) provides an effective therapeutic intervention for treating pathogenesis related to the C9orf72 nucleotide repeat expansions.

### DHX36 Inhibitors

In some embodiments, the technology relates to an inhibitor of DHX36 activity. For instance, embodiments provide an anti-DHX36 antibody, an aptamer, an antisense molecule, a small RNA, a protein, a small molecule, or other inhibitor of DHX36 expression. In some embodiments, the technology comprises use of de novo peptide targeted therapeutics as described, for example, by Chevalier (2017) Nature 550: 74-79, incorporated by reference herein in its entirety.

In some embodiments, the technology relates to a small molecule inhibitor of helicases, e.g., small molecule inhibitors of human helicases. In some embodiments, the technology provides an inhibitor of a DExD/H proteins (e.g., DEAD-Box or DEAH-Box RNA helicase). See, e.g., Radi (2012) “Discovery of the first small molecule inhibitor of human DDX3 specifically designed to target the RNA binding site: Towards the next generation HIV-1 inhibitors” Bioorganic & Medicinal Chemistry Letters 22: 2094-98; and Vesuna (2022) “RK-33, a small molecule inhibitor of host RNA helicase DDX3, suppresses multiple variants of SARS-CoV-2” bioRXiv 2022.02.28.482334, accessed Mar. 28, 2022, each of which is incorporated herein by reference. See also de la Cruz (1999) “Unwinding RNA in Saccharomyces cerevisiae: DEAD-box proteins and related families”. Trends Biochem. Sci. 24 (5): 192-8, incorporated herein by reference.

In some embodiments, the inhibitor of DHX36 activity is a GSEC (G-quadruplex-forming sequence containing lncRNA that binds to DHX36 and inhibits DHX36 G-quadruplex unwinding activity. See, e.g., Matsumura (2017) “The novel G-quadruplex-containing long non-coding RNA GSEC antagonizes DHX36 and modulates colon cancer cell migration” Oncogene 36: 1191, incorporated herein by reference.

In some embodiments, the inhibitor of DHX36 activity is manoalide (4-[3,6-Dihydro-6-hydroxy-5-[4-methyl-6-(2,6,6-trimethyl-1-cyclohexen-1-yl)-3-hexenyl]-2H-pyran-2-yl]-5-hydroxy-2(5H)-furanone; CAS number 75088-80-1), which is a product of marine sponge extracts. See J. Nat. Prod. 2012, 75, 4, 650-654, which is incorporated herein by reference.

In some embodiments, the DHX36 coding sequence and/or a DHX36 messenger RNA is targeted by a CRISPR technology. In some embodiments, the DHX36 coding sequence is targeted by a ribonucleoprotein (RNP) comprising a CRISPR protein (e.g., Cas9 or a protein having the same or similar activity as Cas9) and a gRNA comprising a sequence provided by a portion of the sequence provided by SEQ ID NO: 25 or 26 or a complement of a sequence provided by a portion of the sequence provided by SEQ ID NO: 25 or 26. In some embodiments, a DHX36 messenger RNA is targeted by a ribonucleoprotein (RNP) comprising a CRISPR protein (e.g., Cas13 or a protein having activity that is the same or similar to Cas13) and a gRNA comprising a sequence provided by a portion of the sequence provided by SEQ ID NO: 25 or 26 or a complement of a sequence provided by a portion of the sequence provided by SEQ ID NO: 25 or 26.

In some embodiments, the CRISPR protein is Cas9 or a similar RNA-guided endonuclease having the same on similar activity. Cas9 protein was discovered as a component of the bacterial adaptive immune system (see, e.g., Barrangou et al. (2007) “CRISPR provides acquired resistance against viruses in prokaryotes” Science 315: 1709-1712, incorporated herein by reference). Cas9 is an RNA-guided endonuclease that targets and destroys foreign DNA in bacteria using RNA:DNA base-pairing between a guide RNA (gRNA) and foreign DNA to provide sequence specificity. Recently, Cas9/gRNA complexes (e.g., a Cas9/gRNA RNP) have found use in genome editing (see, e.g., Doudna et al. (2014) “The new frontier of genome engineering with CRISPR-Cas9” Science 346: 6213, incorporated herein by reference); Jinek et al. (2012) “A Programmable Dual-RNA-Guided DNA Endonuclease in Adaptive Bacterial Immunity” Science 337:816-821; and Lee et al. (2016) “The Neisseria meningitidis CRISPR-Cas9 System Enables Specific Genome Editing in Mammalian Cells” Molecular Therapy 24: 645 (2016), each of which is incorporated herein by reference).

In some embodiments, the Cas13 protein is Cas13d, Cas13Rx, another Cas13 protein described herein or known in the art, or a protein having an activity similar to a Cas13 protein such as, e.g., Cas13d, Cas13R, Cas13Rx, or another Cas13 protein described herein or known in the art. See, e.g., Perculija (2021) “Functional Features and Current Applications of the RNA-Targeting Type VI CRISPR-Cas Systems” Adv. Sci. 8: 2004685, incorporated herein by reference. Some non-naturally occurring, engineered CRISPR systems and methods for targeted modification of a nucleic acids and methods for computational identification of CRISPR proteins from nucleotide sequences, such as Cas13 proteins, are described, e.g., in U.S. Pat. App. Pub. No. 2019/0002875, incorporated herein by reference. See, e.g., Abudayyeh (2016) “C2c2 is a single-component programmable RNA-guided RNA-targeting CRISPR effector” Science 353(6299):aaf5573; Konermann (2018) “Transcriptome Engineering with RNA-Targeting Type VI-D CRISPR Effectors” Cell 173: 665-676 e614; Yang (2019) “Dynamic Imaging of RNA in Living Cells by CRISPR-Cas13 Systems” Mol Cell 76: 981-997 e987; Freije (2019) “Programmable Inhibition and Detection of RNA Viruses Using Cas13” Mol Cell 76: 826-837 e811; and Cox (2017) “RNA editing with CRISPR-Cas13” Science 358: 1019-1027, each of which is incorporated herein by reference). See, e.g., Abudayyeh et al. (2017) “RNA targeting with CRISPR-Cas13” Nature 550: 280; Yan (2018) “Cas13d Is a Compact RNA-Targeting Type VI CRISPR Effector Positively Modulated by a WYL-Domain-Containing Accessory Protein” Mol. Cell 70: 327-39, each of which is incorporated herein by reference.

In some embodiments, compositions comprising oligomeric antisense compounds, particularly oligonucleotides, are used to modulate the function of nucleic acid molecules encoding DHX36, ultimately modulating (e.g., decreasing) the amount of DHX36 expressed. This is accomplished by providing antisense compounds that specifically hybridize with one or more nucleic acids encoding DHX36. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of DHX36. In the context of the present disclosure, “modulation” means either an increase (stimulation) or a decrease (inhibition) in the expression of a gone. For example, DHX36 expression may be inhibited to treat or prevent a neurodegenerative disease such as ALS or FTD.

In some embodiments, nucleic acids are small RNAs, for example, siRNAs. “RNA interference (RNAi)” is the process of sequence-specific, post-transcriptional gene silencing initiated by a small interfering RNA (siRNA). During RNAi, siRNA induces degradation of target mRNA with consequent sequence-specific inhibition of gene expression. An “RNA interference,” “RNAi,” “small interfering RNA” or “short interfering RNA” or “siRNA” or “short hairpin RNA” or “shRNA” molecule, or “miRNA” is a RNA duplex of nucleotides that is targeted to a nucleic acid sequence of interest, for example, DHX36. As used herein, the term “siRNA” is a generic term that encompasses all possible RNAi triggers. An “RNA duplex” refers to the structure formed by the complementary pairing between two regions of a RNA molecule. siRNA is “targeted” to a gene in that the nucleotide sequence of the duplex portion of the siRNA is complementary to a nucleotide sequence of the targeted gene. In certain embodiments, the siRNAs are targeted to the sequence encoding DHX36. In some embodiments, the length of the duplex of siRNAs is less than 30 base pairs. In some embodiments, the duplex can be 32, 31, 30, 29, 28, 27, 26, 25, 24, 23, 22, 21, 20, 19, 18, 17, 16, 15, 14, 13, 12, 11 or 10 base pairs in length. In some embodiments, the length of the duplex is 19 to 32 base pairs in length. In certain embodiments, the length of the duplex is 19 or 21 base pairs in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12, 13, 14, 15, 16, 17, 18, 19, 20, 21, 22, 23, 24, 25, 26 or 27 nucleotides in length. In certain embodiments, the loop is 18 nucleotides in length. The hairpin structure can also contain 3′ and/or 5′ overhang portions. In some embodiments, the overhang is a 3′ and/or a 5′ overhang 0, 1, 2, 3, 4 or 5 nucleotides in length.

As used herein, Dicer-substrate RNAs (DsiRNAs) are chemically synthesized asymmetric 25-mer/27-mer duplex RNAs that have increased potency in RNA interference compared to traditional siRNAs. Traditional 21-mer siRNAs are designed to mimic Dicer products and therefore bypass interaction with the enzyme Dicer. Dicer has been shown to be a component of RISC and involved with entry of the siRNA duplex into RISC. Dicer-substrate siRNAs are designed to be optimally processed by Dicer and show increased potency by engaging this natural processing pathway. Using this approach, sustained knockdown has been regularly achieved using sub-nanomolar concentrations. (see, e.g., U.S. Pat. No. 8,084,599; Kim (2005) Nature Biotechnology 23: 222; Rose (2005) Nucleic Acids Res., 33: 4140, each of which is incorporated herein by reference).

The transcriptional unit of a “shRNA” comprises sense and antisense sequences connected by a loop of unpaired nucleotides. shRNAs are exported from the nucleus by Exportin-5, and once in the cytoplasm, are processed by Dicer to generate functional siRNAs. “miRNAs” stem-loops comprise sense and antisense sequences connected by a loop of unpaired nucleotides typically expressed as part of larger primary transcripts (pri-miRNAs), which are excised by the Drosha-DGCR8 complex generating intermediates known as pre-miRNAs, which are subsequently exported from the nucleus by Exportin-5, and once in the cytoplasm, are processed by Dicer to generate functional miRNAs or siRNAs. “Artificial miRNA” or an “artificial miRNA shuttle vector”, as used herein interchangeably, refers to a primary miRNA transcript that has had a region of the duplex stem loop (at least about 9-20 nucleotides) which is excised via Drosha and Dicer processing replaced with the siRNA sequences for the target gene while retaining the structural elements within the stem loop necessary for effective Drosha processing. The term “artificial” arises from the fact the flanking sequences (approximately 35 nucleotides upstream and approximately 40 nucleotides downstream) arise from restriction enzyme sites within the multiple cloning site of the siRNA. As used herein the term “miRNA” encompasses both the naturally occurring miRNA sequences as well as artificially generated miRNA shuttle vectors.

The siRNA can be encoded by a nucleic acid sequence, and the nucleic acid sequence can also include a promoter. The nucleic acid sequence can also include a polyadenylation signal. In some embodiments, the polyadenylation signal is a synthetic minimal polyadenylation signal or a sequence of six Ts.

In some embodiments, the technology provided herein comprises use of any genetic manipulation for use in modulating the expression of DHX36. Examples of genetic manipulation include, but are not limited to, gene knockout (e.g., removing the DHX36 gene from the chromosome using, for example, recombination), expression of antisense constructs with or without inducible promoters, and the like. Delivery of nucleic acid constructs to cells in vitro or in vivo may be conducted using any suitable method. A suitable method is one that introduces the nucleic acid construct into the cell such that the desired event occurs (e.g., expression of an antisense construct).

Introduction of molecules carrying genetic information into cells is achieved by any of various methods including, but not limited to, directed injection of naked DNA constructs, bombardment with gold particles loaded with said constructs, and macromolecule mediated gene transfer using, for example, liposomes, biopolymers, and the like. Exemplary methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer into a variety of solid tumors in animal models and into human solid tumor xenografts in immune-deficient mice. Examples of adenoviral vectors and methods for gene transfer are described in PCT publications WO 00/12738 and WO 00/09675 and in U.S. Pat. Nos. 6,033,908; 6,019,978; 6,001,557; 5,994,132; 5,994,128; 5,994,106; 5,981,225; 5,885,808; 5,872,154; 5,830,730; and 5,824,544, each of which is herein incorporated by reference in its entirety.

Vectors may be administered to subject in a variety of ways. For example, in some embodiments, vectors are administered into tumors or tissue associated with tumors using direct injection. In other embodiments, administration is via the blood or lymphatic circulation (see, e.g., PCT publication 1999/02685, herein incorporated by reference in its entirety). Exemplary dose levels of adenoviral vector are preferably 108 to 1011 vector particles added to the perfusate.

In some embodiments, the technology provides antibodies that inhibit DHX36. Any suitable antibody (e.g., monoclonal, polyclonal, or synthetic) may be utilized in the therapeutic methods disclosed herein. In some embodiments, the antibodies are humanized antibodies. Methods for humanizing antibodies are well known in the art (see, e.g., U.S. Pat. Nos. 6,180,370; 5,585,089; 6,054,297; and 5,565,332; each of which is herein incorporated by reference).

The present technology is not limited to the use of any particular antibody configuration. In some preferred embodiments, the targeting unit is an antigen binding protein. Preferred antigen binding proteins include, but are not limited to, an immunoglobulin, a Fab, F(ab′)2, Fab′ single chain antibody, Fv, single chain (scFv), mono-specific antibody, bi-specific antibody, tri-specific antibody, multivalent antibody, chimeric antibody, humanized antibody, human antibody, CDR-grafted antibody, shark antibody, an immunoglobulin single variable domain (e.g., a nanobody or a single variable domain antibody), minibody, camelid antibody (e.g., from the Camelidae family), microbody, intrabody (e.g., intracellular antibody), and/or de-fucosylated antibody and/or derivative thereof. Mimetics of binding agents and/or antibodies are also provided.

In some embodiments, scFv polypeptides described herein are fused to Fe regions to generate minibodies. As used herein, the term “fragment crystallizable region (Fe region)” refers to the tail region of an antibody that interacts with cell surface receptors called Fc receptors and some proteins of the complement system. This property allows antibodies to activate the immune system. In IgG, IgA, and IgD antibody isotypes, the Fc region comprises two identical protein fragments, derived from the second and third constant domains of the antibody's two heavy chains; IgM and IgE Fc regions contain three heavy chain constant domains (CH domains 2-4) in each polypeptide chain. The Fc regions of IgGs bear a highly conserved N-glycosylation site.

In some embodiments, the Fe region is derived from an IgG. In some embodiments, the IgC is human IgG1, although other suitable Fc regions derived from other organisms or antibody frameworks may be utilized.

In some embodiments, scFv polypeptides described herein are fused to chimeric antigen receptors. Chimeric antigen receptors (CARs), (also known as chimeric immunoreceptors, chimeric T cell receptors, artificial T cell receptors or CAR-T) are engineered receptors, which graft an arbitrary specificity onto an immune effector cell (T cell). Typically, these receptors are used to graft the specificity of an antibody (e.g., an scFv described herein) onto a T cell, with transfer of their coding sequence facilitated by retroviral vectors. The receptors are called chimeric because they are composed of parts from different sources.

Further, the present technology also envisages expression vectors comprising nucleic acid sequences encoding any of the above polypeptides or fusion proteins thereof or functional fragments thereof, as well as host cells expressing such expression vectors. Suitable expression systems include constitutive and inducible expression systems in bacteria or yeasts, virus expression systems, such as baculovirus, semliki forest virus and lentiviruses, or transient transfection in insect or mammalian cells. Suitable host cells include E. coli, Lactococcus lactis, Saccharomyces cerevisiae, Schizosaccharomyces pombe, Pichia pastoris, and the like. Suitable animal host cells include HEK 293, COS, S2, CHO, NSO, DT40 and the like. The cloning, expression, and/or purification of the antibodies can be done according to techniques known by the skilled person in the art.

It will be understood that polypeptides described herein may be identified with reference to the nucleotide and/or amino acid sequence corresponding to the variable and/or complementarity determining regions (“CDRs”) thereof.

Also within the scope of the technology are natural or synthetic analogs, mutants, variants, alleles, homologs, and orthologs (herein collectively referred to as “variants”) of the immunoglobulin single variable domains of the technology as defined herein. Thus, according to one embodiment of the technology, the term “immunoglobulin single variable domain” in its broadest sense also covers such variants, in particular variants of the antibodies described herein. Generally, in such variants, one or more amino acid residues may have been replaced, deleted, and/or added compared to the antibodies of the technology as defined herein. Such substitutions, insertions, or deletions may be made in one or more of the framework regions and/or in one or more of the CDRs. Variants, as used herein, are sequences wherein each or any framework region and each or any complementarity determining region shows at least 80% identity, preferably at least 85% identity, more preferably 90% identity, even more preferably 95% identity or, still even more preferably 99% identity with the corresponding region in the reference sequence (e.g., FR1_variant versus FR1_reference, CDR1_variant versus CDR1_reference, FR2_variant versus FR2_reference, CDR2_variant versus CDR2_reference, FR3_variant versus FR3_reference, CDR3_variant versus CDR3_reference, FR4_variant versus FR4_reference), as can be measured electronically by making use of algorithms such as PILEUP and BLAST. (See, e.g., Higgins & Sharp, CABIOS 5: 151 (1989); Altschul S. F., W. Gish, W. Miller, E. W. Myers, D. J. Lipman. Basic local alignment search tool. J. Mol. Biol. 1990; 215:403-10.) Software for performing BLAST analyses is publicly available through the National Center for Biotechnology Information (on the worldwide web at ncbi.nlm.nih.gov). Such variants of immunoglobulin single variable domains may be of particular advantage since they may have improved potency or other desired properties.

A “deletion” is defined herein as a change in either amino acid or nucleotide sequence in which one or more amino acid or nucleotide residues, respectively, are absent as compared to an amino acid sequence or nucleotide sequence of a parental polypeptide or nucleic acid. Within the context of a protein, a deletion can involve deletion of about two, about five, about ten, up to about twenty, up to about thirty or up to about fifty or more amino acids. A protein or a fragment thereof may contain more than one deletion.

An “insertion” or “addition” is a change in an amino acid or nucleotide sequence that has resulted in the addition of one or more amino acid or nucleotide residues, respectively, as compared to an amino acid sequence or nucleotide sequence of a parental protein. “Insertion” generally refers to addition of one or more amino acid residues within an amino acid sequence of a polypeptide, while “addition” can be an insertion or refer to amino acid residues added at an N- or C-terminus, or both termini. Within the context of a protein or a fragment thereof, an insertion or addition is usually of about one, about three, about five, about ten, up to about twenty, up to about thirty, or up to about fifty or more amino acids. A protein or fragment thereof may contain more than one insertion.

A “substitution,” as used herein, results from the replacement of one or more amino acids or nucleotides by different amino acids or nucleotides, respectively as compared to an amino acid sequence or nucleotide sequence of a parental protein or a fragment thereof. It is understood that a protein or a fragment thereof may have conservative amino acid substitutions which have substantially no effect on the protein's activity. By conservative substitutions is intended combinations such as gly, ala; val, ile, leu, met; asp, glu; asn, gin; ser, thr; lys, arg; cys, met; and phe, tyr, trp.

By means of non-limiting examples, a substitution may, for example, be a conservative substitution (as described herein) and/or an amino acid residue may be replaced by another amino acid residue that naturally occurs at the same position (e.g., for an antibody, in another variable domain). Thus, any one or more substitutions, deletions or insertions, or any combination thereof, that either improve the properties of the antibody of the technology or that at least do not effectively detract from the desired properties or from the balance or combination of desired properties of the antibody of the technology (e.g., to the extent that the antibody is no longer suited for its intended use) are included within the scope of the technology. A skilled person will generally be able to determine and select suitable substitutions, deletions or insertions, or suitable combinations of thereof, based on the disclosure herein and optionally after a limited degree of routine experimentation, which may, for example, involve introducing a limited number of possible substitutions and determining their influence on the properties of the antibodies thus obtained.

Further, depending on the host organism used to express the immunoglobulin single variable domain of the technology, such deletions and/or substitutions may be designed in such a way that one or more sites for post-translational modification (such as one or more glycosylation sites) are removed, as will be within the ability of the person skilled in the art. Alternatively, substitutions or insertions may be designed so as to introduce one or more sites for attachment of functional groups (as described herein), for example, to allow site-specific pegylation.

Examples of modifications, as well as examples of amino acid residues within the immunoglobulin single variable domain, that can be modified (e.g., either on the protein backbone but preferably on a side chain), methods and techniques that can be used to introduce such modifications, and the potential uses and advantages of such modifications will be clear to the skilled person. For example, such a modification may involve the introduction (e.g., by covalent linking or in another suitable manner) of one or more functional groups, residues or moieties into or onto the immunoglobulin single variable domain of the technology, and in particular of one or more functional groups, residues or moieties that confer one or more desired properties or functionalities to the immunoglobulin single variable domain of the technology. Examples of such functional groups and of techniques for introducing them will be clear to the skilled person, and can generally comprise all functional groups and techniques mentioned in the general background art cited hereinabove as well as the functional groups and techniques known for the modification of pharmaceutical proteins, and in particular for the modification of antibodies or antibody fragments (including ScFvs and single domain antibodies), for which reference is, for example, made to Remington's Pharmaceutical Sciences, 16th ed., Mack Publishing Co., Easton, Pa. (1980). Such functional groups may, for example, be linked directly (for example, covalently) to an immunoglobulin single variable domain of the technology, or optionally via a suitable linker or spacer, as will again be clear to the skilled person. One of the most widely used techniques for increasing the half-life and/or reducing immunogenicity of pharmaceutical proteins comprises attachment of a suitable pharmacologically acceptable polymer, such as poly(ethyleneglycol) (PEG) or derivatives thereof (such as methoxypoly(ethyleneglycol) or mPEG). Generally, any suitable form of pegylation can be used, such as the pegylation used in the art for antibodies and antibody fragments (including but not limited to (single) domain antibodies and ScFvs); reference is made to, for example, Chapman, Nat. Biotechnol., 54, 531-545 (2002); by Veronese and Harris, Adv. Drug Deliv. Rev. 54, 453-456 (2003), by Harris and Chess, Nat. Rev. Drug. Discov., 2, (2003) and in Int'l Pat. Pub. No. WO04060965. Various reagents for pegylation of proteins are also commercially available, for example, from Nektar Therapeutics, USA. Preferably, site-directed pegylation is used, in particular via a cysteine-residue (see, for example, Yang et al., Protein Engineering, 16, 10, 761-770 (2003). For example, for this purpose, PEG may be attached to a cysteine residue that naturally occurs in an antibody of the technology, an antibody of the technology may be modified so as to suitably introduce one or more cysteine residues for attachment of PEG, or an amino acid sequence comprising one or more cysteine residues for attachment of PEG may be fused to the N- and/or C-terminus of a antibody of the technology, all using techniques of protein engineering known per se to the skilled person. Preferably, for the immunoglobulin single variable domains and proteins of the technology, a PEG is used with a molecular weight of more than 5000, such as more than 10,000 and less than 200,000, such as less than 100,000; for example, in the range of 20,000-80,000.

Another, usually less preferred modification comprises N-linked or O-linked glycosylation, usually as part of co-translational and/or post-translational modification, depending on the host cell used for expressing the immunoglobulin single variable domain or polypeptide of the technology. Another technique for increasing the half-life of an immunoglobulin single variable domain may comprise the engineering into bifunctional constructs or into fusions of immunoglobulin single variable domains with peptides (for example, a peptide against a serum protein such as albumin).

Yet another modification may comprise the introduction of one or more detectable labels or other signal-generating groups or moieties, depending on the intended use of the labeled antibody. Suitable labels and techniques for attaching, using, and detecting them will be clear to the skilled person and, for example, include, but are not limited to, fluorescent labels (such as fluorescein, isothiocyanate, rhodamine, phycoerythrin, phycocyanin, allophycocyanin, o-phthaldehyde, and fluorescamine and fluorescent metals such as Eu or others metals from the lanthanide series), phosphorescent labels, chemiluminescent labels or bioluminescent labels (such as luminal, isoluminol, theromatic acridinium ester, imidazole, acridinium salts, oxalate ester, dioxetane or GFP and its analogs), radio-isotopes, metals, metals chelates or metallic cations or other metals or metallic cations that are particularly suited for use in in vivo, in vitro or in situ diagnosis and imaging, as well as chromophores and enzymes (such as malate dehydrogenase, staphylococcal nuclease, delta-V-steroid isomerase, yeast alcohol dehydrogenase, alpha-glycerophosphate dehydrogenase, triose phosphate isomerase, biotinavidin peroxidase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholine esterase). Other suitable labels will be clear to the skilled person and, for example, include moieties that can be detected using NMR or ESR spectroscopy. Such labeled antibodies and polypeptides of the technology may, for example, be used for in vitro, in vivo or in situ assays (including immunoassays known per se such as ELISA, RIA, EIA and other “sandwich assays,” etc.), as well as in vivo diagnostic and imaging purposes, depending on the choice of the specific label.

As will be clear to the skilled person, another modification may involve the introduction of a chelating group, for example, to chelate one of the metals or metallic cations referred to above. Suitable chelating groups, for example, include, without limitation, diethyl-enetriaminepentaacetic acid (DTPA) or ethylenediaminetetraacetic acid (EDTA). Yet another modification may comprise the introduction of a functional group that is one part of a specific binding pair, such as the biotin-(strept)avidin binding pair. Such a functional group may be used to link the antibody of the technology to another protein, polypeptide or chemical compound that is bound to the other half of the binding pair, e.g., through formation of the binding pair. For example, an antibody of the technology may be conjugated to biotin, and linked to another protein, polypeptide, compound or carrier conjugated to avidin or streptavidin. For example, such a conjugated antibody may be used as a reporter, for example, in a diagnostic system where a detectable signal-producing agent is conjugated to avidin or streptavidin. Such binding pairs may, for example, also be used to bind the antibody of the technology to a carrier, including carriers suitable for pharmaceutical purposes. One non-limiting example are the liposomal formulations described by Cao and Suresh, Journal of Drug Targeting, 8, 4, 257 (2000). Such binding pairs may also be used to link a therapeutically active agent to the antibody of the technology.

In some embodiments, the immunoglobulin single variable domain of the present technology is fused to a detectable label, either directly or through a linker. Preferably, the detectable label is a radio-isotope or radioactive tracer, which is suitable for medical applications, such as in in vivo nuclear imaging. Examples include, without the purpose of being limitative, 99mTc, 123I, 125I, 111In, 18F, 64Cu, 67Ga, 68Ga, and any other radio-isotope which can be used in animals, in particular mouse or human.

In some embodiments, the immunoglobulin single variable domain of the present technology is fused to a moiety selected from the group consisting of a toxin, or to a cytotoxic drug, or to an enzyme capable of converting a prodrug into a cytotoxic drug, or to a radionuclide, or coupled to a cytotoxic cell, either directly or through a linker.

In some embodiments, the present technology provides an antibody-drug conjugate and/or an antibody-enzyme conjugate. In certain embodiments, the antibody drug conjugates are administered to cells expressing DHX36.

As used herein, “linkers” are peptides of 1 to 50 amino acids length and are typically chosen or designed to be unstructured and flexible. These include, but are not limited to, synthetic peptides rich in Gly, Ser, Thr, Gln, Glu or further amino acids that are frequently associated with unstructured regions in natural proteins. (See, e.g., Dosztanyi Z., V. Csizmok, P. Tompa, and I. Simon (2005). IUPred: web server for the prediction of intrinsically unstructured regions of proteins based on estimated energy content. Bioinformatics (Oxford, England), 21(16), 3433-4.)

In some embodiments, the therapeutic polypeptide is an immunoglobulin or fragment thereof. Examples include, but are not limited to, aptamers and immunoglobulins. Immunoglobulins (antibodies) are proteins generated by the immune system to provide a specific molecule capable of complexing with an invading molecule commonly referred to as an antigen. Natural antibodies have two identical antigen-binding sites, both of which are specific to a particular antigen. The antibody molecule recognizes the antigen by complexing its antigen-binding sites with areas of the antigen termed epitopes. The epitopes fit into the conformational architecture of the antigen-binding sites of the antibody, enabling the antibody to bind to the antigen.

The immunoglobulin molecule is composed of two identical heavy and two identical light polypeptide chains, held together by interchain disulfide bonds. Each individual light and heavy chain folds into regions of about 110 amino acids, assuming a conserved three-dimensional conformation. The light chain comprises one variable region (termed VL) and one constant region (CL), while the heavy chain comprises one variable region (VH) and three constant regions (CH1, CH2 and CH3). Pairs of regions associate to form discrete structures. In particular, the light and heavy chain variable regions, VL and VH, associate to form an “FV” area that contains the antigen-binding site.

The variable regions of both heavy and light chains show variability in structure and amino acid composition from one antibody molecule to another, whereas the constant regions show little variability. Each antibody recognizes and binds an antigen through the binding site defined by the association of the heavy and light chain, variable regions into an FV area. The light-chain variable region VL and the heavy-chain variable region VH of a particular antibody molecule have specific amino acid sequences that allow the antigen-binding site to assume a conformation that binds to the antigen epitope recognized by that particular antibody.

Within the variable regions are found regions in which the amino acid sequence is extremely variable from one antibody to another. Three of these so-called “hypervariable” regions or “complementarity-determining regions” (CDRs) are found in each of the light and heavy chains. The three CDRs from a light chain and the three CDRs from a corresponding heavy chain form the antigen-binding site.

Cleavage of naturally occurring antibody molecules with the proteolytic enzyme papain generates fragments that retain their antigen-binding site. These fragments, commonly known as Fabs (for Fragment, antigen binding site) are composed of the CL, VL, CH1, and VH regions of the antibody. In the Fab, the light chain and the fragment of the heavy chain are covalently linked by a disulfide linkage.

Monoclonal antibodies against target antigens (e.g., DHX36) are produced by a variety of techniques including conventional monoclonal antibody methodologies such as the somatic cell hybridization techniques of Köhler and Milstein, Nature, 256:495 (1975). Although in some embodiments, somatic cell hybridization procedures are preferred, other techniques for producing monoclonal antibodies are contemplated as well (e.g., viral or oncogenic transformation of B lymphocytes). A preferred animal system for preparing hybridomas is the murine system.

Hybridoma production in the mouse is a well-established procedure. Immunization protocols and techniques for isolation of immunized splenocytes for fusion are known in the art. Fusion partners (e.g., murine myeloma cells) and fusion procedures are also known.

Human monoclonal antibodies (mAbs) directed against human proteins can be generated using transgenic mice carrying the complete human immune system rather than-the mouse system. Splenocytes from the transgenic mice are immunized with the antigen of interest, which are used to produce hybridomas that secrete human mAbs with specific affinities for epitopes from a human protein. (See e.g., WO 91/00906; WO 91/10741; WO 92/03918; WO 92/03917 (each of which is herein incorporated by reference in its entirety); N. Lonberg et al., Nature, 368:856-859 [1994]; L. L. Green et al., Nature Genet., 7:13-21 [1994]; S. L. Morrison et al., Proc. Nat. Acad. Sci. USA, 81:6851-6855 [1994]; Bruggeman et al., Immunol., 7:33-40 [1993]; Tuaillon et al., Proc. Nat. Acad. Sci. USA, 90:3720-3724 [1993]; and Bruggernan et al. Eur. J. Immunol., 21:1323-1326 [1991]).

Monoclonal antibodies can also be generated by other methods known to those skilled in the art of recombinant DNA technology. An alternative method, referred to as the “combinatorial antibody display” method, has been developed to identify and isolate antibody fragments having a particular antigen specificity, and can be utilized to produce monoclonal antibodies. (See e.g., Sastry et al., Proc. Nat. Acad. Sci. USA, 86:5728 [1989]; Huse et al., Science, 246:1275 [1989]; and Orlandi et al., Proc. Nat. Acad. Sci. USA, 86:3833 [1989]). After immunizing an animal with an immunogen as described above, the antibody repertoire of the resulting B-cell pool is cloned. Methods are generally known for obtaining the DNA sequence of the variable regions of a diverse population of immunoglobulin molecules by using a mixture of oligomer primers and the PCR. For instance, mixed oligonucleotide primers corresponding to the 5′ leader (signal peptide) sequences and/or framework 1 (FR1) sequences, as well as primer to a conserved 3′ constant region primer can be used for PCR amplification of the heavy and light chain variable regions from a number of murine antibodies. (See e.g., Larrick et al., Biotechniques, 11:152-156 [1991]). A similar strategy can also be used to amplify human heavy and light chain variable regions from human antibodies (See e.g., Larrick et al., Methods: Companion to Methods in Enzymology, 2:106-110 [1991]).

The term modified antibody is also intended to include antibodies, such as monoclonal antibodies, chimeric antibodies, and humanized antibodies which have been modified by, for example, deleting, adding, or substituting portions of the antibody. For example, an antibody can be modified by deleting the hinge region, thus generating a monovalent antibody. Any modification is within the scope of the technology so long as the antibody has at least one antigen binding region specific.

Chimeric mouse-human monoclonal antibodies can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted. (See e.g., Robinson et al., PCT/US86/02269; European Patent Application 184,187; European Patent Application 171,496; European Patent Application 173,494; WO 86/01533; U.S. Pat. No. 4,816,567; European Patent Application 125,023 [each of which is herein incorporated by reference in its entirety]; Better et al., Science, 240:1041-1043 [1988]; Liu et al., Proc. Nat. Acad. Sci. USA, 84:3439-3443 [1987]; Liu et al., J. Immunol., 139:3521-3526 [1987]; Sun et al., Proc. Nat. Acad. Sci. USA, 84:214-218 [1987]; Nishimura et al., Canc. Res., 47:999-1005 [1987]; Wood et al., Nature, 314:446-449 [1985]; and Shaw et al., J. Natl. Cancer Inst., 80:1553-1559 [1988]).

The chimeric antibody can be further humanized by replacing sequences of the Fv variable region that are not directly involved in antigen binding with equivalent sequences from human Fv variable regions. General reviews of humanized chimeric antibodies are provided by S. L. Morrison, Science, 229:1202-1207 (1985) and by Oi et al., Bio. Techniques, 4:214 (1986). Those methods include isolating, manipulating, and expressing the nucleic acid sequences that encode all or part of immunoglobulin Fv variable regions from at least one of a heavy or light chain.

Suitable humanized antibodies can alternatively be produced by CDR substitution (e.g., U.S. Pat. No. 5,225,539 (incorporated herein by reference in its entirety); Jones et al., Nature, 321:552-525 [1986]; Verhoeyan et al., Science, 239:1534 [1988]; and Beidler et al., J. Immunol., 141:4053 [1988]). All of the CDRs of a particular human antibody may be replaced with at least a portion of a non-human CDR or only some of the CDRs may be replaced with non-human CDRs. It is only necessary to replace the number of CDRs required for binding of the humanized antibody to the Fe receptor.

An antibody can be humanized by any method that is capable of replacing at least a portion of a CDR of a human antibody with a CDR derived from a non-human antibody. The human CDRs may be replaced with non-human CDRs; using oligonucleotide site-directed mutagenesis.

Also within the scope of the technology are chimeric and humanized antibodies in which specific amino acids have been substituted, deleted or added. In particular, preferred humanized antibodies have amino acid substitutions in the framework region, such as to improve binding to the antigen. For example, in a humanized antibody having mouse CDRs, amino acids located in the human framework region can be replaced with the amino acids located at the corresponding positions in the mouse antibody. Such substitutions are known to improve binding of humanized antibodies to the antigen in some instances.

The antibodies can be of various isotypes, including, but not limited to: IgG (e.g., IgG1, IgG2, IgG2a, IgG2b, IgG2c, IgG3, IgG4); IgM; IgA1; IgA2; IgAsec; IgD; and IgE. In some preferred embodiments, the antibody is an IgG isotype. In other preferred embodiments, the antibody is an IgM isotype. The antibodies can be full-length (e.g., an IgG1, IgG2, IgG3, or IgG4 antibody) or can include only an antigen-binding portion (e.g., a Fab, F(ab′)2, Fv or a single chain Fv fragment).

In preferred embodiments, the immunoglobulin is a recombinant antibody (e.g., a chimeric or a humanized antibody), a subunit, or an antigen binding fragment thereof (e.g., has a variable region, or at least a complementarity determining region (CDR)). In some embodiments, the immunoglobulin is monovalent (e.g., includes one pair of heavy and light chains, or antigen binding portions thereof). In other embodiments, the immunoglobulin is a divalent (e.g., includes two pairs of heavy and light chains, or antigen binding portions thereof).

The present disclosure further provides pharmaceutical compositions (e.g., comprising the compounds described above). The pharmaceutical compositions of the present disclosure may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral, or parenteral. Parenteral administration includes intravenous, intra-arterial, subcutaneous, intraperitoneal, or intramuscular injection or infusion; or intracranial, e.g., intrathecal or intraventricular, administration. In certain embodiments, DHX36 inhibitors and agents are administered by methods that bypass the BBB including, for example, direct application to the surface of the CNS, to the parenchyma of the CNS, to the ventricles of the CNS, and to the cerebrospinal fluid (CSF) of the CNS. In particular, intrathecal and epidural administration may be achieved by single shot, a series of single shots, and/or by continuous administration to the CSF. In certain embodiments, continuous administration to the CSF is provided by a programmable external pump. In other embodiments, continuous administration is provided by a programmable implantable pump.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present disclosure include, but are not limited to, solutions, emulsions, and liposome containing formulations. These compositions may be generated from a variety of components that include, but are not limited to, preformed liquids, self emulsifying solids and self emulsifying semisolids.

The pharmaceutical formulations of the present disclosure, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present disclosure may be formulated into any of many possible dosage forms such as, but not limited to, tablets, capsules, liquid syrups, soft gels, suppositories, and enemas. The compositions of the present disclosure may also be formulated as suspensions in aqueous, non aqueous or mixed media. Aqueous suspensions may further contain substances that increase the viscosity of the suspension including, for example, sodium carboxymethylcellulose, sorbitol and/or dextran. The suspension may also contain stabilizers.

Agents that enhance uptake of oligonucleotides at the cellular level may also be added to the pharmaceutical and other compositions of the present disclosure. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present disclosure may additionally contain other adjunct components conventionally found in pharmaceutical compositions. Thus, for example, the compositions may contain additional, compatible, pharmaceutically active materials such as, for example, antipruritics, astringents, local anesthetics or anti inflammatory agents, or may contain additional materials useful in physically formulating various dosage forms of the compositions of the present disclosure, such as dyes, flavoring agents, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC50s found to be effective in in vitro and in vivo animal models or based on the examples described herein. In general, dosage is from 0.01 g to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the composition is administered in maintenance doses, ranging from 0.01 μg to 100 g per kg of body weight, once or more daily, to once every 20 years.

The technology also relates to methods of treating a subject with a drug appropriate for the subject's malady. According to another aspect of the technology, a method is provided for treating a subject in need of such treatment with an effective amount of a compound or a salt thereof. The method involves administering to the subject an effective amount of a compound or a salt thereof in any one of the pharmaceutical preparations described above, detailed herein, and/or set forth in the claims. The subject can be any subject in need of such treatment. In the foregoing description, the technology is in connection with a compound or salts thereof. Such salts include, but are not limited to, bromide salts, chloride salts, iodide salts, carbonate salts, and sulfate salts. It should be understood, however, that the compound is a member of a class of compounds and the technology is intended to embrace pharmaceutical preparations, methods, and kits containing related derivatives within this class. Another aspect of the technology then embraces the foregoing summary but read in each aspect as if any such derivative is substituted wherever “compound” appears.

In some embodiments, a subject is tested to assess the presence, the absence, or the level of a malady and/or a condition (e.g., ALS, FTD). Such testing is performed, e.g., by detecting, assaying, or measuring a biomarker (e.g., DHX36 helicase, DHX36 helicase activity, a hexanucleotide (GGGGCC) repeat expansion in the C9orf72 gene, an RNA comprising a hexanucleotide (GGGGCC) repeat expansion, an RNA comprising a G-quadruplex), a metabolite, a physical symptom, an indication, etc., to determine the risk of or the presence of the malady or condition. In some embodiments, the subject is treated with a compound described herein based on the outcome of the test. In some embodiments, a subject is treated, a sample is obtained and the level of detectable agent is measured, and then the subject is treated again based on the level of detectable agent that was measured. In some embodiments, a subject is treated, a sample is obtained and the level of detectable agent is measured, the subject is treated again based on the level of detectable agent that was measured, and then another sample is obtained and the level of detectable agent is measured. In some embodiments, other tests (e.g., not based on measuring the level of detectable agent) are also used at various stages, e.g., before the initial treatment as a guide for the initial dose. In some embodiments, a subsequent treatment is adjusted based on a test result, e.g., the dosage amount, dosage schedule, identity of the drug, etc. is changed. In some embodiments, a patient is tested, treated, and then tested again to monitor the response to therapy and/or change the therapy. In some embodiments, cycles of testing and treatment may occur without limitation to the pattern of testing and treating, the periodicity, or the duration of the interval between each testing and treatment phase. As such, the technology contemplates various combinations of testing and treating without limitation, e.g., test/treat, treat/test, test/treat/test, treat/test/treat, test/treat/test/treat, test/treat/test/treat/test, test/treat/test/test/treat/treat/treat/test, treat/treat/test/treat, test/treat/treat/test/treat/treat, etc.

Although the disclosure herein refers to certain illustrated embodiments, it is to be understood that these embodiments are presented by way of example and not by way of limitation.

### EXAMPLES

During the development of embodiments of the technology described herein, experiments were conducted, and data were collected indicating that an RNA helicase, DHX36, binds and unfolds G-quadruplexes formed by C9orf72 repeat RNAs in vitro and that endogenous DHX36 interacts with C9orf72 repeat RNAs in vivo. Furthermore, data indicated that reducing DHX36 amount and/or activity significantly decreased the levels of DPRs in C9orf72 ALS patient-derived induced pluripotent stem cells (iPSCs) and in motor neurons. In sum, the data indicated that DHX36 positively regulates the production of DPR by resolving C9orf72 repeat RNA G-quadruplexes, thus indicating a mechanism for the pathogenesis of relevant neurological disorders.

### Materials and Methods

DNA constructs for translation reporters. For cell-based translation reporters, a pcDNA3.1 backbone construct expressing EGFP under the CMV promoter and NeoR under the SV40 promoter was first generated. The EGFP coding sequence was amplified with a forward primer producing NheI, EcoRI, and HindIII sites and a reverse primer producing an XhoI site. The resulting fragment was ligated into pcDNA3.1 (ThermoFisher) between the Nhe I and XhoI sites. The repeat sequence (GGGGCC)4, (GGGGCC)s, or their respective length-matched non-G-quadruplex-forming controls were synthesized (Integrated DNA Technologies) and ligated into the pcDNA3.1-EGFP backbone construct between the NheI and HindIII sites. The (GGGGCC)28 DNA fragment was generated using self-templating PCR as we previously described (25) and inserted into the EcoRI site of pcDNA3.1-EGFP. A length-matched non-G-quadruplex-forming control for (GGGGCC)28 was amplified from a luciferase gene and ligated into pcDNA3.1-EGFP between the EcoRI and HindIII sites. For the in vitro translation assays, (GGGGCC)4-EGFP, (GGGGCC)s-EGFP, and their length-matched controls were amplified from the abovementioned pcDNA3.1 constructs and subcloned into pF3A WG(BYDY) (Promega) between the NcoI and SacI sites. The sequences of the control oligonucleotides are provided in Table 1.

TABLE 1
Oligonucleotides

| Name | Sequence (5′ to 3′) | SEQ ID NO: |
|---|---|---|
| r(GGGGCC)8-Cy5 | rGrGrGrGrCrCrGrGrGrGrCrCrGrGrGrGrCrCrGrGrGrGrCr | 1 |
| | CrGrGrGrGrCrCrGrGrGrGrCrCrGrGrGrGrCrCrGrGrGrGrC | |
| | rC/3cy5Sp/ | |
| r(GGGGCC)4-Cy5 | rGrGrGrGrCrCrGrGrGrGrCrCrGrGrGrGrCrCrGrGrGrGrCr | 2 |
| | C/3cy5Sp/ | |
| r(GTGTCC)4-Cy5 | rGrUrGrUrCrCrGrUrGrUrCrCrGrUrGrUrCrCrGrUrGrUrCr | 3 |
| | C/3cy5Sp/ | |
| r(CCCCGG)4-Cy5 | rCrCrCrCrGrGrCrCrCrCrGrGrCrCrCrCrGrGrCrCrCrCrGr | 4 |
| | G/3Cy5Sp/ | |
| Cy5-d(GGGGCC)4 | /5cy5Sp/GGGGCCGGGGCCGGGGCCGGGGCC | 5 |
| Cy5-d(CCCCGG)4 | /5cy5Sp/CCCCGGCCCCGGCCCCGGCCCCGG | 6 |
| (C4G2)4-Cy3 | CCCCGGCCCCGGCCCCGGCCCCGG/3Cy3Sp/ | 7 |
| Non-GQ-24-mer | AGAGAATTCTGGAGGTGGCGGTTC | 8 |
| Non-GQ-48-mer | AGAGAATTCTGAAGATGCCAAAAACATTAAGTCTGGAGGTGGCGGTT | 9 |
| | C | |
| Non-GQ-168-mer | GGAAGATGCCAAAAACATTAAGAAGGGCCCAGCGCCATTCTACCCAC | 10 |
| | TCGAAGACGGGACCGCCGGCGAGCAGCTGCACAAAGCCATGAAGCGC | |
| | TACGCCCTGGTGCCCGGCACCATCGCCTTTACCGACGCACATATCGA | |
| | GGTGGACATTACCTACGCCGAGTACTT | |
| Nluc-qF | GTCCGTAACTCCGATCCAAAG | 11 |
| Nluc-qR | TGCCATAGTGCAGGATCACCT | 12 |
| Fluc-qF | GTGACTTCCCATTTGCCACC | 13 |
| Fluc-qR | TGATCTGGTTGCCGAAGATG | 14 |
| EGFP-qF | GACGTAAACGGCCACAAGTT | 15 |
| EGFP-qR | AAGTCGTGCTGCTTCATGTG | 16 |
| GAPDH-qF | GTCTCCTCTGACTTCAACAGCG | 17 |
| GAPDH-qR | ACCACCCTGTTGCTGTAGCCAA | 18 |
| ACTB-qF | CACCATTGGCAATGAGCGGTTC | 19 |
| ACTB-qR | AGGTCTTTGCGGATGTCCACGT | 20 |
| NeoR-qF | ACCTTGCTCCTGCCGAGAAAGTAT | 21 |
| NeoR-qR | ATGTTTCGCTTGGTGGTCGAATGG | 22 |

Protein purification and electrophoretic mobility shift, assay. DHX36 was purified as previously described (27). Briefly, a variant of DHX36 (Δ1-56, EKK146AAA) containing a N terminal GST-tag and a C-terminal poly-histidine tag was expressed in Escherichia coli LOBSTR (DE3) cells. The variant was used because it has wild-type-like G-quadruplex-binding and unwinding activities but shows enhanced solubility and resistance to proteolysis during protein purification. The cell cultures were induced by the addition of 1 mM IPTG and incubated overnight at 20° C. The DHX36 variant was first purified using a Ni-NTA Superflow column (Qiagen), which was immediately followed by a GSTrap 4B3 (GE Healthcare) column. The eluted protein was incubated overnight at 4° C. with TEV protease for removal of the GST-tag. The following day, the protein sample was applied to a size-exclusion chromatography column (GE Healthcare) equilibrated with 25 mM HEPES-KOH pH 7.5, 150 mM KCl, 10% Glycerol, and 0.5 mM TCEP (tris(2-carboxyethyl)phosphine). Fractions corresponding to DHX36 were collected and stored at 4° C. For electrophoretic mobility shift assays (EMSA), RNA oligonucleotides 3′-labelled with Cy5 (0.2 μM) were heated in annealing buffer (10 mM Tris-HCl pH 7.4, 50 mM KCl or LiCl, and 1 mM EDTA) at 95° C. for 5 minutes and slowly cooled to 25° C. The annealed RNA oligonucleotides (20 nM) were then incubated with DHX36 at the indicated concentrations in binding reaction buffer (100 mM Tris-HCl pH 7.4, 100 mM KCl or LiCl, 1 mM EDTA, 1 mM DTT, 5% Glycerol, 2 ng/μl yeast tRNA, 0.01% BSA (w/v), and 5 U/μl RiboLock RNase Inhibitor (Thermo Scientific)) at 25° C. for 20 minutes. The mixture was then resolved on a 10% non-denaturing polyacrylamide gel (Acrylamide/Bis 37.5:1) in 1×TBE buffer with 75 mM KCl. DNA EMSAs follow the same protocol above without RNase inhibitors in the binding buffer or yeast tRNA but instead with 15 ng/μl Sheared Salmon Sperm DNA (Ambion, AM9680). Images were captured with Amersham Typhoon Imager 9200 and analyzed with Image Studio version 5.2 (L1-COR Biosciences). The binding curve fittings were generated by GraphPad Prism 8 using nonlinear regression.

G-quadruplex-Hemm colorimetric Assay. RNA oligonucleotides (5 μM) were annealed slowly in buffer containing 10 mM Tris-HCl (pH 7.4) and 100 mM KCl from 95° C. to 25° C. 20 μl of the RNA oligonucleotide solution was then transferred to a final 50 μl reaction buffer (25 mM HEPES-NaOH pH 7.4, 100 mM KCl, 2 mM MgCl₂, 0.05% Triton X-100, 1% DMSO and 2 μM RNA oligonucleotides) containing 5 μM Hemin. After an incubation at 25° C. for 30 minutes, another 50 μl of the reaction buffer containing 5 mM ABTS and 0.8 mM H₂O₂ was added to trigger the colorimetric reaction. The absorption spectra of the reaction were recorded by BIOTEK SYNERGY H4 Hybrid Microplate Reader. For the unwinding test, DHX36 or BSA was added together with the RNA in the reaction buffer to incubate for 15 minutes followed by the addition of 5 μM Hemin for another incubation at 25° C. for 15 minutes.

Single-molecule fluorescence resonance energy transfer data acquisition. Single-molecule fluorescence resonance energy transfer (smFRET) data were acquired using a custom-built prism-type total internal reflection (TIR) inverted fluorescence microscope (Olympus IX 71) as described previously (28-29). The substrate was annealed in 10 mM Tris-IICl (pII 7.5) and 100 mM KCl to make a partial duplex RNA:

(SEQ ID NO: 23)
rUrGrGrCrGrArCrGrGrCrArGrCrGrArGrGrCrGrGrGrGrCrCrG
rGrGrGrCrCrGrGrGrGrCrCrGrGrGrGrCrCrUrUrUrUrUrUrUrU
rUrU/Cy3/
(SEQ ID NO: 24)
/Cy5/rGrCrCrUrCrGrCrUrGrCrCrGrUrCrGrCrCrA/biotin/

The partial duplex RNA was immobilized on the PEG-passivated surface coated with neutravidin (50 μg/ml). The smFRET measurements were carried out in an imaging buffer containing 10 mM Tris-HCl (pH 7.5), 100 mM KCl, 1 mM MgCl₂, 10% glycerol, 0 or 1 mM ATP, and an oxygen scavenging system (1 mg/ml glucose oxidase, 0.5% (w/v) glucose, 4 μg/ml catalase, and 10 mM Trolox). A solid-state 532 nm diode laser (Compass 315M, Coherent) was used to excite the Cy3 (donor). The fluorescence from Cy3 and Cy5 (acceptor) were simultaneously collected using a water immersion objective, projected onto the EMCCD camera (Andor), and donor and acceptor signals split through the dichroic mirror (cutoff=630 nm). Before and after addition of purified DHX36, all data were recorded with 100 ms frame integration time, processed by IDL script, and analyzed by Matlab scripts (30).

Cell culture and transfection. Both HEK293 cells and HeLa Flp-In cells were cultured in DMEM supplemented with 10% (v/v) fetal bovine serum at 37° C. with 5% CO₂. The C9orf72 RAN translation reporter assay was performed as described previously (31). Briefly, HeLa Flp-In cells were plated in 6-well plates at 8×10⁵ cells per well, followed by treatment with 2 μg/ml doxycycline for 24 hours the following day. Then, the cells were harvested to measure the NLuc and FLuc luciferase activity by The Nano-Glo Dual-Luciferase Reporter Assay System (Promega) on BIOTEK SYNERGY H1 Hybrid Microplate Reader. For transfection on HEK293 cells, cells were plated in 12-well plates at 4×10⁵ cells per well and transfected with 500 ng of plasmid per well with 1 μl of Lipofectamine 2000 (Invitrogen). 24 hours after the transfection, cells were harvested for quantitative RT-PCR and immunoblotting analyses.

Lentivirus-mediatedgene knockdown. For lentivirus production, the lentiviral vector pLKO.1 expressing shRNAs against DHX36 (clone ID: TRCN0000050704, TRCN0000050705; Broad Institute) or a control pLKO.1 vector expressing a non-targeting shRNA was co-transfected with the packaging plasmid psPAX2 (Addgene #12260) and the envelope plasmid pMD2.G (Addgene #12259) into HEK293 cells at a molar ratio of 1:1:1. Fresh medium was changed 6 hours after the transfection, and cells were allowed to continue to grow for 48 hours. Supernatants containing the virus were then collected and filtered through 0.45 μm membrane (Millipore Sigma HVHP02500). The filtered supernatants were mixed with 4× Lentivirus Concentrator Solution (1×PBS (pH 7.4) and 40% PEG-8000 (w/v)) and left at 4° C. overnight. The resulting solution was centrifuged at 1600×g for 60 minutes at 4° C.; the virus pellets were resuspended into cold PBS in 1/20 of the original volume and then aliquoted for storage. To generate stable DHX36 knockdown cell lines, HEK293 cells and HeLa Flp-In cells were transduced with 20× concentrated lentiviruses 24 hours after cell seeding in culture plates. Puromycin (2 μg/ml) was added at 48 hours after transduction and cells were selected with the drug for 7 days before harvesting.

Reverse transcription and quantitative PCR. RNA was isolated by RNeasy Plus Mini kit (Qiagen), followed by reverse transcription using QuantiTect Reverse Transcription Kit (Qiagen) according to the manufacturer protocol. Quantitative PCR was carried out with PowerUp SYBR Green Master Mix (Thermo Scientific) using a C1000 Touch thermal cycler with a CFX96 Real-Time System (BioRad). The qPCR primer sequences are provided in Table 1.

Human tissues and western blotting. Human spinal cord tissues used in this study are described in Table 2.

TABLE 2
Patient tissues

| Sample | | | Clinical | ALS | Age of | | |
|---|---|---|---|---|---|---|---|
| No. | Patient ID | Source | diagnosis | pathology | sampling | Gender | Region |
| 1 | 90015 | VABBB | CTRL | Non | 66 | M | SC-C |
| 2 | 90018 | VABBB | CTRL | Non | 82 | M | SC-C |
| 3 | 100012 | VABBB | CTRL | Non | 81 | F | SC-C |
| 4 | 120016 | VABBB | CTRL | Non | 63 | F | SC-C |
| 5 | 95 | JHMI | CTRL | Non | 72 | M | SC-C |
| 6 | 103 | JHMI | CTRL | Non | 22 | M | SC-C |
| 7 | 100007 | VABBB | fALS- | Yes | 61 | M | SC-C |
| | | | C9orf72 | (TDP-43 | | | |
| | | | | positive) | | | |
| 8 | 38 | JHMI | sALS- | Yes | 34 | F | SC-C |
| | | | C9orf72 | | | | |
| 9 | 88 | JHMI | fALS- | Yes | 59 | M | SC-C |
| | | | C9orf72, | | | | |
| | | | FTD | | | | |
| 10 | 92 | JHMI | fALS- | Yes | 72 | M | SC-C |
| | | | C9orf72 | | | | |
| 11 | NEUTP019MY9 | BNI | ALS- | Yes | 62 | F | SC-C |
| | | | C9orf72 | | | | |

Abbreviations: ALS (Amyotrophic Lateral Sclerosis, fALS (Familial Amyotrophic Lateral Sclerosis), sALS (Sporadic Amyotrophic Lateral Sclerosis), FTD (Frontotemporal Dementia), CTRL (Control), M (Male), F (Female), JHMI (Johns Hopkins Medical Institute), VABBB (VA Biorepository Brain Bank), BNI (Barrow Neurological Insititute), SC-C (Spinal Cord-Cervical).

For immunoblot analysis, spinal cords were rinsed with cold PBS and homogenized with cold RIPA buffer using glass tissue grinders. The homogenates were then centrifuged at 4° C. at 1,000×g for 10 minutes, and the supernatants were centrifuged again at 16,000×g for 10 minutes, with the final supernatant used for immunoblot analysis.

Protein samples from tissues or cells were resolved on 12% SDS-PAGE gels and then transferred to nitrocellulose membranes (Bio-rad). The membranes were blocked in 5% milk in 1×TBST (Tris-Buffered Saline, 0.1% Tween-20) at room temperature for 1 hour, followed by incubation with a primary antibody in 5% BSA in 1×TBST containing 0.03% sodium azide at 4° C. overnight. The primary antibodies include anti-DHX36 (Abcam, ab70269, 1:5000), anti-GFP (Invitrogen, GF28R, 1:500), anti-6-Tubulin (Cell Signaling, #2128, 1:1000), anti-GAPDH (Thermo Scientific, TAB1001, 1:2000) and anti-6-Actin (Santa Cruz, sc-47778, 1:1000). Membranes were washed three times with 1×TBST for 5 minutes at room temperature and then incubated with a secondary antibody diluted 1:1000 in blocking buffer at room temperature for 2 hours. The secondary antibodies include goat anti-rabbit IgG IRDye (800 CW, 926-32211) and donkey anti-mouse IgG (680 LT, 926-68022). Membranes were again washed three times with 1×TBST at room temperature for 5 minutes and then imaged on the Odyssey system and analyzed with Image Studio version 5.2 (LI-COR).

In vitro transcription and translation. The pF3A WG (BYDY) plasmids containing GGGGCC repeats or control sequences were linearized by XbaI and purified using the Wizard SV Gel and PCR Clean-Up system (Promega). The linearized plasmids were then used as templates for transcription by a HiScribe T7 transcription kit (NEB, E2030S). Following DNase treatments, the transcription products were purified using RNA Clean & Concentrator-25 (ZYMO RESEARCH) and examined by 5% denaturing urea polyacrylamide gel electrophoresis. In vitro translation assays were performed using a wheat germ extract kit (Promega, L4380). Briefly, reactions (50 μl) were carried out in 96-well black plates (Costar) containing 25 μl wheat germ extract, 1.25 μg template RNA, 0.08 mM amino acid mixture, 50 mM potassium acetate, and 0.8 U/μl RiboLock RNase Inhibitor (Thermo Scientific). The fluorescence signal was recorded by a BIOTEK SYNERGY H1 microplate reader with the following kinetic setting: temperature 25° C., excitation 480 nm, emission 507 nm, running time 1.5 hours, kinetic interval 2 minutes, shake linear for 1 s; read speed normal, optics position top, read height 5 mm.

RNA immunoprecipitation. RNA immunoprecipitation was performed as described with modifications (32). HeLa Flp-In cells were seeded in 10 cm dishes at 7×10⁶ cells/dish and treated with 2 μg/ml doxycycline for 24 hours the following day. Cells were then harvested and rinsed once with cold PBS. The cell pellets were resuspended in 0.5 ml cytoplasmic lysis buffer (25 mM HEPES pH 7.9, 5 mM KCl, 0.5 mM MgCl2, and 0.5% IGEPAL CA-630 (v/v)) supplemented with protease inhibitors (cOmplete, EDTA-free cocktail, Roche) and 1.6 U/μl of Ribolock RNase inhibitors (Thermo Scientific), left on ice for 20 minutes, and centrifuged at 2500× g for 5 minutes at 4° C. The supernatants were set aside on ice and the cell pellets were resuspended in 0.5 ml nuclear lysis buffer (25 mM Hepes, pH 7.9, 10% sucrose (w/v), 350 mM NaCl, and 0.01% IGEPAL CA-630 (v/v)) supplemented with protease inhibitors (cOmplete, EDTA-free cocktail, Roche) and 1.6 U/μl of Ribolock RNase in-hibitors (Thermo Scientific). To lyse the nuclei, the tubes were vortexed for 30 s followed by a 30-minute incubation at 4° C. with end-over-end mixing. The nuclear and cytoplasmic fractions were combined, and the insoluble material was removed by centrifugation at 20,000×g for 10 minutes at 4° C. The cell lysates were then precleared with 10 μl equilibrated Pierce Protein A/C Magnetic Beads (Thermo Scientific) at 4° C. for 30 minutes. For 1 ml of the precleared cell lysate, 50 μl was taken for RNA input and 50 μl for protein input. For immunoprecipitations, 2.5 pg of anti-DHX36 antibody (ab70269, Abcam) or Normal Rabbit IgG (Cell Signaling Technology) was added to 450 μl cell lysate and incubated at 25° C. for 1 hour with end-over-end mixing. Then 5 μl equilibrated Pierce Protein A/G Magnetic Beads was added to each sample for another 1 hour of mixing at 25° C. Beads were washed 9 times with a 1:1 mixture of cytoplasmic and nuclear lysis buffers followed by RNA isolation with RNeasy Plus Mini Kit (Qiagen). The isolated RNA was then reverse transcribed by QuantiTect Reverse Transcription Kit (Qiagen) for qPCR using PowerUp SYBR Green Master Mix (Thermo Scientific) according to the manufacturer protocols.

RNA fluorescence in situ hybridization. RNA fluorescence in situ hybridization was performed as previously reported with modifications (33). Briefly, cells grown on coverslips (Deckglaser, Germany) were fixed in 3.75% formaldehyde in PBS for 10 minutes at room temperature, followed by permeabilization in ice cold 70% ethanol for 30 minutes. After rehydration in wash buffer (40% deionized formamide in 2×SSC) for 10 minutes, cells were prehybridized at 55° C. for 10 minutes in hybridization buffer (40% deionized formamide, 2×SSC, 20 μg/mL BSA, 100 mg/mL dextran sulfate, 10 μg/mL yeast tRNA, and 2 mM Vanadyl Sulfate Ribonucleosides). Cells were then incubated at 55° C. for 2 hours in hybridization buffer containing 125 nM (CCCCGG)4-Cy3 probe, which was denatured at 95° C. for 5 minutes before being added to the buffer, followed by washing three times with wash buffer at 55° C. for 10 minutes. Coverslips were then stained with 0.5 pg/mL DAPI (4′,6-diamidino-2-phenylindole) in PBS at room temperature for 10 minutes and mounted with ProLong Gold Antifade (Thermo Fisher Scientific). Images were obtained using a Leica SP8 confocal microscope with 0.5 μm z-step size and matched exposure settings. All solutions were made with DEPC-treated water and formamide was freshly used.

Human iPSC and motor neuron cultures. iPSCs were grown in StemFlex medium (Thermo Scientific, A3349401) on Matrigel-(Corning, 354230) coated plates. To generate motor neurons, iPSCs were differentiated as previously described (34). Briefly, iPSCs were first differentiated into neuroepithelial progenitor (NEP) cells using neural medium (1:1 DMEM/F12:neurobasal medium, glutamax, N2 supplement, B27 supplement, and ascorbic acid), supplemented with 3 μM CHIR99021, 2 μM SB431542, and 2 μM DMH-1 for 6 days. NEP cells were then split and grown in neural medium supplemented with 1 μM CHIR99021, 2 μM SB431542, 2 μM DMH-1, 0.1 μM retinoic acid (RA), and 0.5 μM purmorphamine to generate motor neuron progenitor (MNP) cells. To generate motor neuron-like cells, MNPs were disassociated by cell scraper and placed in suspension culture using neural medium supplemented with 0.5 μM RA and 0.1 μM purmorphamine. After 6 days, motor neuron-like cells were disassociated and plated onto Matrigel-coated plates. Mature motor neurons were generated in 12 days using neural medium supplemented with 0.5 μM RA, 0.1 μM purmorphamine, and 0.1 μM compound E, with medium change every other day. To generate stable shRNA-expressing iPSCs, 50 μl of concentrated lentivirus solution (shRNA clone ID: TRCN0000050704) was added to each well of iPSCs cultured in 6-well plates. Following 2 days of transduction, cells were cultured in selection medium containing 1 μg/ml puromycin for 7 days before analysis. For transduction of iMNs, lentiviruses were added at day 7 of the motor neuron maturing stage and incubated for 2 days. After another 8 days of culturing, motor neurons were harvested for analysis. Human iPSCs used in this study were described in Table 3.

TABLE 3
Induced pluripotent stem cells

| Sample | | | Clinical | | | |
|---|---|---|---|---|---|---|
| No. | Subject ID | Genotype | Status | Gender | Age | Source |
| 1 | NDS00241 | — | Control | M | 36 | NINDS |
| 2 | NDS00242 | — | Control | F | 49 | NINDS |
| 3 | NDS00246 | C9orf72 | At risk | F | 61 | NINDS |
| 4 | ND10689 | C9orf72 | Affected | F | 51 | NINDS |

NINDS: National Institute of Neurological Disease and Stroke;
M: male;
F: female

Poly-GPELA. Cells were lysed in RIPA buffer (Sigma, R0278) containing 1× proteinase inhibitor (Roche, cOm-plete, EDTA-free) and centrifuged at 16000×g for 20 minutes at 4° C. The supernatants were collected, and protein concentrations were quantified with the Pierce BCA protein assay (Thermo Scientific). The samples were then diluted to the concentration of 1 mg/ml for ELISA as previously described (21). Briefly, 0.375 μg/mL biotinylated rabbit anti-GP antibody was incubated in 96-well small spot streptavidin coated plates for 1 hour at room temperature. Following three PBST washes, 35 μL cell lysate was added per well in duplicate and incubated for a 3 hours at room temperature. After three PBST washes, sulfo-tagged detection antibody was then added at 1 μg/mL and incubated for 1 hour. Following another set of PBST washes, 150 μL read buffer was added and samples immediately imaged by MESO Quick-Plex SQ 120. Specificity was verified using lysates of HEK293 cells overexpressing GFP-tagged dipeptide repeat proteins. All reagents were from Meso Scale Discovery (MSD).

Statistical analysis. Statistical analyses were performed using GraphPad Prism 8. Quantitative analysis was per-formed using two-tailed Student's t-test. P value less than to 0.05 was taken as statistically significant. The n values and other details of statistical analysis are described in the descriptions of the drawings.

### Example 1—DHX36 Helicase has High Affinity for G-Quadruplexes Formed by C9orf72 Repeat RNA

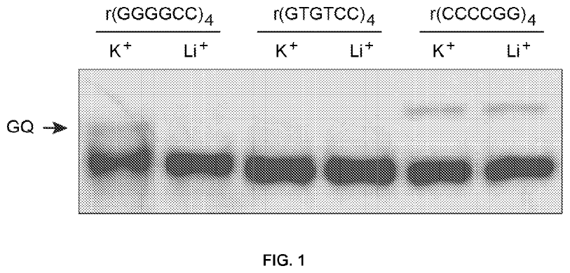

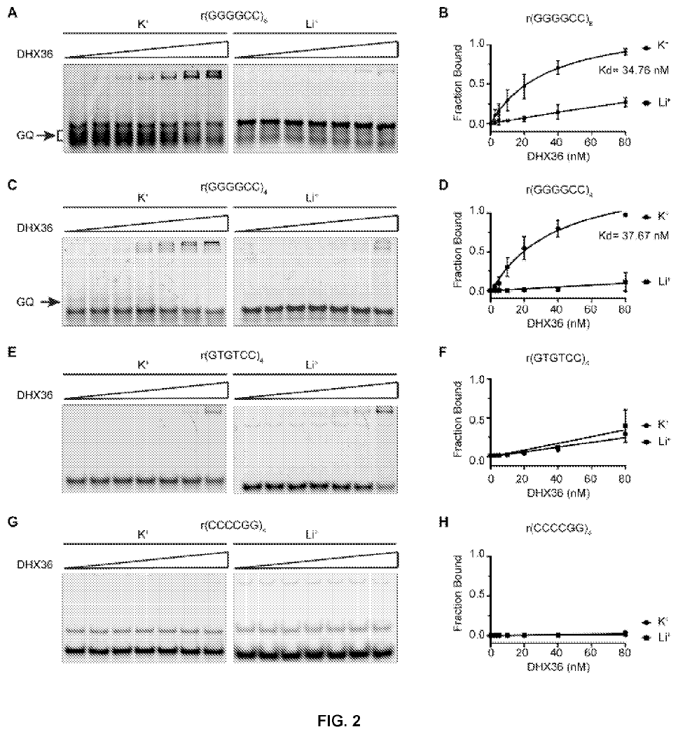

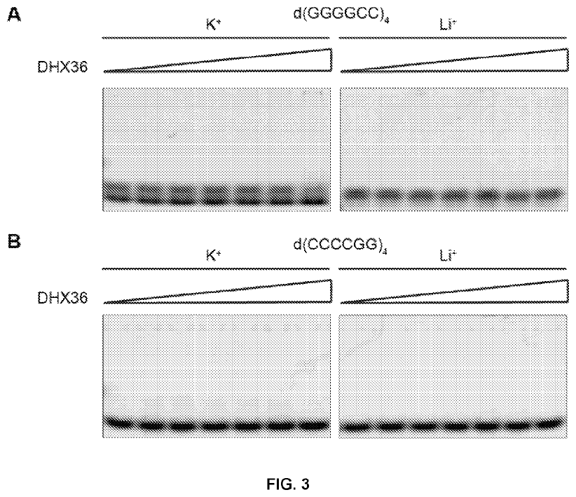

C9orf72 GGGGCC repeat RNA can form parallel G-quadruplexes that exhibit high thermal stability (23, 25, 26, 35). Accordingly, experiments were conducted to verify that a helicase that resolves the RNA G-quadruplex structure has a regulatory role in the cellular functions associated with the C9orf72 GGGGCC repeat RNAs. As a member of the DEAH/RHA family of helicases, DHX36 shows an extremely high affinity for DNA and RNA G-quadruplexes, and it preferentially unwinds parallel G-quadruplexes (27, 36). During the development of embodiments of the technology described herein, experiments were conducted to examine if DHX36 acts on r(GGGGCC)n G-quadruplexes. In these experiments, EMSAs were used to determine whether the various structures of C9orf72 repeat RNAs are recognized by DHX36. It has previously been shown that four repeats of the G4C2 RNA are sufficient to form stable G-quadruplexes (25). Since K+ stabilizes G-quadruplex structures better than does Li+ (37), the formation of r(GGGGCC)4 G-quadruplexes is more likely induced by the presence of K+, whereas the formation of hairpins is more likely induced by r(GGGGCC)4 Li+ (FIG. 1). Data collected from the EMSA assay indicated that DHX36 preferentially bound the G-quadruplex conformation of r(GGGGCC)4 or r(GGGGCC)8; in particular, DHX36 was detected to have a much higher affinity for the RNAs under K+ conditions than under Li+ conditions (FIG. 2A-FIG. 2D). Notably, r(GGGGCC)8 formed more stable multiple G-quadruplexes than did r(GGGGCC)4, as indicated on electrophoresis gels by smeared bands and a relatively high proportion of upper-shifted bands for r(GGGGCC)8 in the presence of K+ when compared to Li+ (FIG. 2A, FIG. 2C), indicating that the longer C9orf72 repeat RNA adopts multiple G-quadruplex conformations that increase its stability and complexity (see, e.g., 23). To confirm the specificity of DHX36 binding for r(GGGGCC)4 G-quadruplexes, mutations were introduced into the repeat RNA that disabled the formation of the G-quadruplex structure. The mutated repeat RNA, r(GTGTCC)4, did not exhibit any K+-dependent binding to DHX36 (FIG. 2E, FIG. 2F), indicating that the interaction between the helicase and the repeat RNAs was mediated by the G-quadruplex. In addition, it was found that another control, the C9orf72 antisense repeat RNA r(CCCCGG)4, which cannot form G-quadruplexes (38), showed no interaction with DHX36 (FIG. 2G, FIG. 2H). Notably, unlike parallel RNA G-quadruplexes, single stranded RNAs are bound by DHX36 but with a much lower affinity (FIG. 2E). Furthermore, DHX36 has little preferential binding toward the d(GGGGCC)4 DNA G-quadruplexes (FIG. 3A, FIG. 3B), which adopts an antiparallel orientation (25, 39) and thus indicating that DHX36 favors binding parallel G-quadruplexes (36). Taken together, these data indicate that DHX36 can specifically bind G-quadruplexes of C9orf72 G4C2 repeat RNA.

### Example 2—DHX36 Unwinds the G-Quadruplex Structure Formed by C9orf72 Repeat RNA

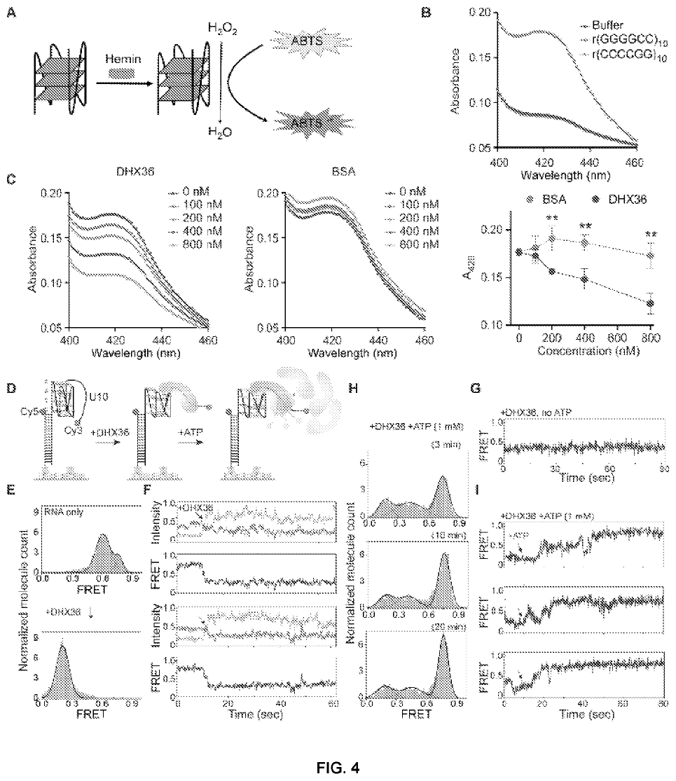

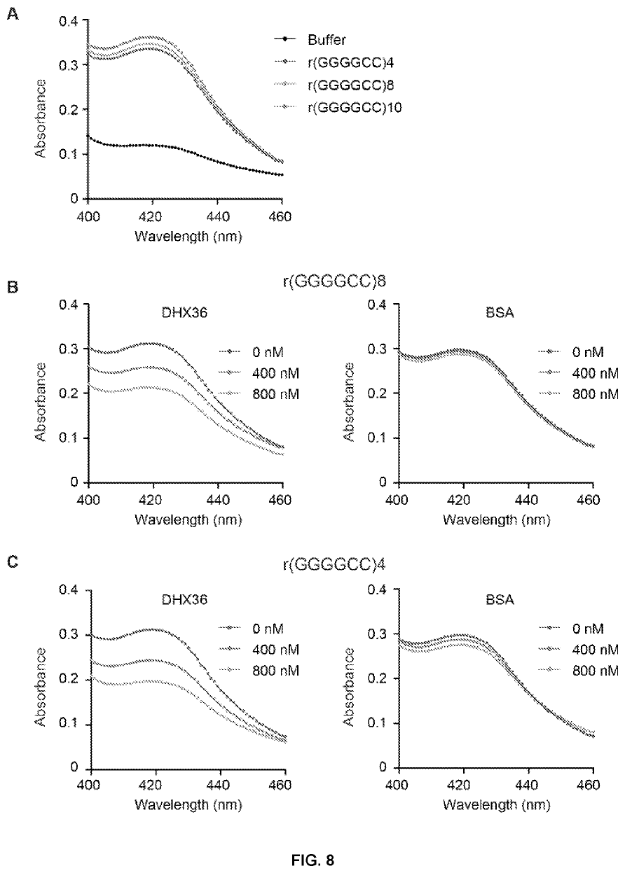

After establishing the binding of DHX36 to the C9orf72 repeat RNA G-quadruplex, experiments were conducted to investigate the helicase activity of DHX36 on the G-quadruplex structure. First, a G-quadruplex-hemin RNAzyme system was used to monitor the integrity of the G-quadruplex structure. G-quadruplexes are known to be able to associate with hemin (iron(III)-protoporphyrin IX) to form peroxidase-mimicking enzymes, whose activity is dependent on an intact G-quadruplex structure (40, 41). To test the ability of C9orf72 repeat RNA to catalyze peroxidase-mimic reactions, ABTS (2,2′-azino-bis(3-ethylbenzothiazoline-6-sulfonic acid)) was used as a substrate. The G-quadruplexes, once folded from C9orf72 repeat RNAs, bind hemin with high affinity and form RNAzyme complexes to catalyze the oxidation of ABTS, yielding a peroxidation product, ABTS+, which has a maximum absorption around 412-422 nm (FIG. 4A). As expected, r(GGGGCC)10 showed strong G-quadruplex-mediated production of ABTS+, exhibiting an absorption maximum around 420 nm (FIG. 4B). In contrast, the antisense repeat r(CCCCGG)10, known to form intramolecular A-form-like helices (38), showed a significantly lower signal that was similar to or identical to that of the negative buffer control, indicating a lack of peroxidase activity (FIG. 4B). Using the G-quadruplex-hemin RNAzyme system, experiments were conducted to examine the effect of DHX36 on the G-quadruplexes formed by r(GGGGCC)10. When DHX36 was added to the reaction mixture, the amount of ABTS+ decreased significantly as the concentration of DHX36 increased (FIG. 4C), indicating that DHX36 bound to the G-quadruplex-hemin complex and altered its conformation and activity. As a control for the DHX36 protein, the addition of bovine serum albumin (BSA) protein to the reaction had no major effect on the activity of the G-quadruplex-hemin RNAzyme, confirming the specificity of DHX36 activity. In addition, experiments were conducted to test the effects of DHX36 on the RNAzyme activities of r(GGGGCC)4 and r(GGGGCC)8, which showed similar results as those of r(GGGGCC)10 (FIG. 8).

To obtain further insight into DHX36 helicase activity toward the C9orf72 repeat RNA G-quadruplexes, single-molecule fluorescence resonance energy transfer (smFRET) experiments were performed. A GGGGCC-containing RNA construct labelled with Cy3 and Cy5 was provided for smFRET imaging. The RNA had four GGGGCC repeats and a poly 15 rU10 tail for DHX36 loading at the 3′ end (FIG. 4D). FRET values were collected from >4000 molecules in 20 different fields of view to construct a FRET histogram. The G4C2-containing RNA alone yielded the main FRET peak at ˜0.6, consistent with the expected distance between Cy3 and Cy5 separated by the G4C2 repeat and U10 tail (FIG. 4E). Upon addition of 10 nM DHX36, the mid-FRET peak shifted to low FRET at ˜0.2, indicating that all RNA molecules are occupied by DHX36 (FIG. 4E). The representative smFRET traces displayed a decrease in FRET signals, indicating the partial disruption of G-quadruplex structures by DHX36 binding (FIG. 4F). The DHX36 remains bound as indicate by the data showing a steady low FRET state (FIG. 4G). When 1 mM ATP was added to the DHX36-bound G4C2-U10 substrate, the FRET histogram gradually shifted back to high values (FIG. 4H), and the smFRET traces displayed slow FRET fluctuations that transitioned to the high-FRET state (FIG. 4I). This pattern represents ATP-dependent repetitive unfolding of G-quadruplexes by DHX36 followed by dislodging of DHX36, in accordance with previous observations of the action of DHX36 on other model G-quadruplex substrates (42, 43). In summary, the data indicate that DHX36 binds G-quadruplex structure formed by C9orf72 repeat RNA with high affinity and unfolds the G-quadruplex structure by hydrolyzing ATP.

### Example 3—DHX36 Positively Regulates C9orf72 Repeat-Associated RAN Translation

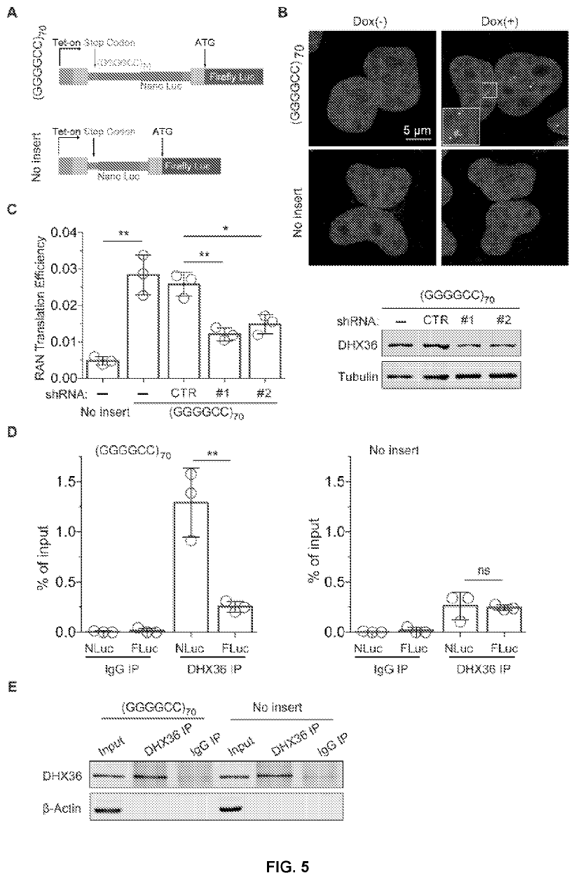

Given the strong binding of DHX36 protein to the C9orf72 repeat RNA and its effects on the RNA structures, experiments were conducted to test whether DHX36 plays a role in repeat RNA-mediated cellular processes. One of the unique consequences of C9orf72 hexanucleotide repeat expansion is repeat-associated non-AUG-dependent (RAN) translation. To investigate a potential role for DHX36 in this non-canonical translation, experiments used a system comprising inducible dual-luciferase-based reporter cell lines designed for monitoring C9orf72 RAN translation (FIG. 5A) (31). Briefly, a (GGGGCC)70 repeat was inserted upstream of the ATG-less coding sequence of the NanoLuc luciferase (NLuc) in the poly-GP frame to induce RAN translation, and the firefly luciferase (FLuc) with an ATG start codon was cloned in the same construct as an internal control for canonical translation. The reporter cassette, with or without the (GGGGCC)70 repeat, was integrated into the unique Flp-In site in the stable reporter HeLa cell lines (31). Using an RNA fluorescence in situ hybridization (FISH) assay with the (CCCCGG)4 probe, data collected during the experiments confirmed the expression of the GGGGCC repeat RNA in the cells harboring the (GGGGCC)70 repeats (FIG. 5B). In particular, these data included observations of repeat RNA foci in the nucleus, which is a pathological hallmark in C9orf72-related ALS patients (44). As expected, the (GGGGCC)70 repeat induced significant RAN translation as measured by the NLuc expression in the poly-GP frame relative to the FLuc expression; the no-insert control gave only a minimal background signal (FIG. 5C). To test the role of DHX36 in RAN translation, experiments were conducted to stably knock down the helicase in the (GGGGCC)70 reporter cell line by using two different lentivirus-mediated shRNAs. In accordance with the reduction of the DHX36 protein, the level of RAN translation, as indicated by the NLuc signal relative to FLuc, was significantly reduced (FIG. 5C), demonstrating that DHX36 positively regulates C9orf72 RAN translation.

During the development of embodiments of the technology described herein, experiments were conducted to verify that DHX36 regulates C9orf72 RAN translation by interacting with repeat RNA and to validate further the in vitro observation of direct binding of DHX36 to GGGGCC repeat RNAs. In particular, native RNA immunoprecipitation experiments were performed in the cell-based system. Endogenous DHX36 was pulled down by incubating a specific antibody with whole cell lysates from the (GGGGCC)70 or no-repeat reporter lines, and RNA associated with DHX36 was co-immunoprecipitated for further quantitative analysis. When compared with a non-specific IgG control, there was an enrichment of DHX36 protein from the pull-down samples, indicating the specific immunoprecipitation of DHX36 protein (FIG. 5E). Quantitative reverse transcription and PCR analysis showed that the NLuc RNA fused with (GGGGCC)70 RNA was significantly enriched in the pull-down samples when compared to the FLuc RNA control (FIG. 5D). In contrast, the NLuc RNA without the (GGGGCC)70 flanking sequence from the no-insert control cells had only a minimal background signal, comparable to that of FLuc RNA. The nearly 5-fold enrichment of (GGGGCC)70 repeat-containing RNA confirmed that DHX36 binds the C9orf72 repeat RNA in vivo (FIG. 5D). Taken together, these data indicate that DHX36 interacts with C9orf72 repeat RNA and positively regulates its RAN translation.

### Example 4—DHX36 Releases Translational Repression Caused by C9orf72 Repeat RNA G-Quadruplexes

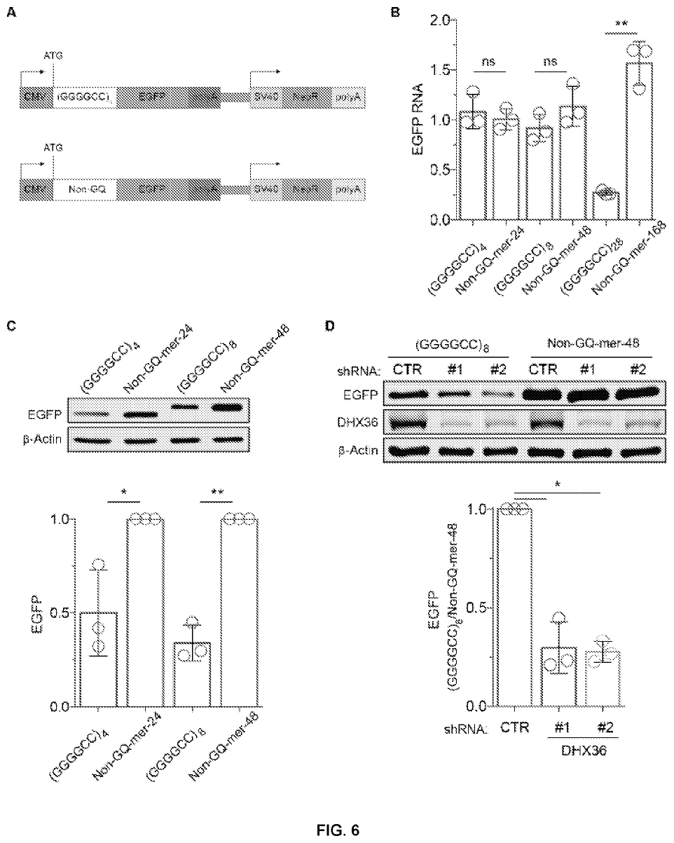

Given the reported role of G-quadruplexes in inhibiting translation elongation (45), experiments were conducted to verify that DHX36 regulates C9orf72 RAN translation by resolving the G-quadruplexes in repeat RNA and thereby relieving the translational repression. A series of reporter constructs were produced in which GGGGCC repeats were placed between the ATG start codon and the EGFP coding sequence (FIG. 6A). Since C9orf72 hexanucleotide repeat expansion can impair transcription in a G-quadruplex-dependent manner (25), experiments tested different lengths of GGGGCC repeats to minimize the effect of impaired transcription on the translation of the reporter gene. For example, when a segment of 28 hexanucleotide repeats, (GGGGCC)28, was engineered into the reporter, the level of EGFP mRNA as measured by quantitative RT-PCR was significantly reduced when compared to those with shorter repeats; however, when the repeats were replaced with the non-G-quadruplex-forming sequence, the EGFP mRNA signal reverted to normal levels (FIG. 6B), indicating that the G-quadruplex structures formed by (GGGGCC)28 led to a reduction in the transcription of the reporter gene. By comparison, for the reporters containing (GGGGCC)4 or (GGGGCC)8, the levels of EGFP mRNA did not change when the repeats were replaced with the non-G-quadruplex-forming sequences (FIG. 6B), indicating that the transcription of the reporter gene was not affected by the secondary structures of these shorter hexanucleotide repeats. Therefore, reporters containing (GGGGCC)4 or (GGGGCC)8 were used to examine the effects of G-quadruplexes and DHX36 protein on translation in the absence of the confounding effects of G-quadruplex struc-tures on transcription.

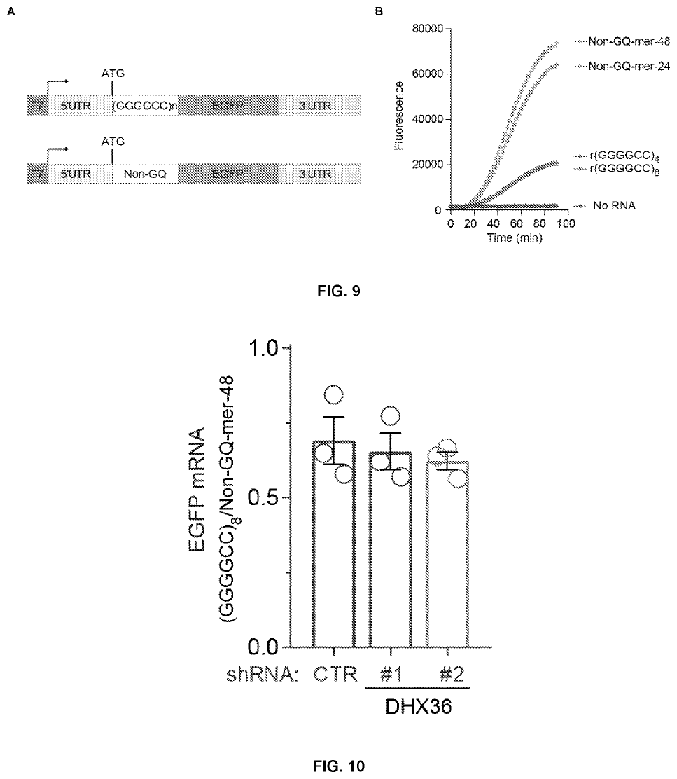

Although (GGGGCC)4 or (GGGGCC)8 did not affect the mRNA levels of the reporter gene, these repeats significantly reduced the levels of the EGFP reporter protein when compared to the non-G-quadruplex-forming control sequence (FIG. 6C, FIG. 6D). Since these hexanucleotide repeats are positioned downstream of the start codon, this finding indicates that the G-quadruplex structures formed by these repeats inhibit translation elongation, leading to a reduced level of protein product. Further, a wheat germ extract-based in vitro system was developed for monitoring translation in real time by placing the EGFP coding sequence immediately following the GGGGCC repeats and then measuring the translation rates based on the EGFP fluorescence. Consistent with the observations in HEK293 cells, the reporters containing either (GGGGCC)4 or (GGGGCC)8 showed lower translation rates than did the controls with the GGGGCC repeats replaced by non-G-quadruplex-forming sequences of equal lengths (FIG. 9A, FIG. 9B), further confirming that the GGGGCC repeats impaired translation.

Next, to examine whether DHX36 plays a role in the hexanucleotide repeat-dependent regulation of translation, stable DHX36 knockdown HEK293 cells were generated using shRNA-expressing lentivirus. Data indicated that the levels of EGFP protein expressed from the reporter containing (GGGGCC)8 were significantly reduced after DHX36 knockdown when compared to the EGFP protein levels in a non-targeting shRNA control (FIG. 6D). Since EGFP mRNA levels were not changed (FIG. 10), this result indicates that endogenous DHX36 promotes translation elongation over C9orf72 repeat RNA. Furthermore, when (GGGGCC)8 was replaced with a randomized, non-G-quadruplex-forming control sequence of the same length, no effect of DHX36 knockdown on the reporter gene translation was detected (FIG. 6D), consistent with the observation that DHX36 recognizes and resolves the G-quadruplex structure of GGGGCC repeat RNA. Taken together, these data indicate that DHX36 unwinds the G-quadruplexes of C9orf72 repeat RNA and promotes translation elongation through the repeat RNA sequences.

### Example 5—DHX36 Facilitates Native RAN Translation and is Up-Regulated in Patient Tissues

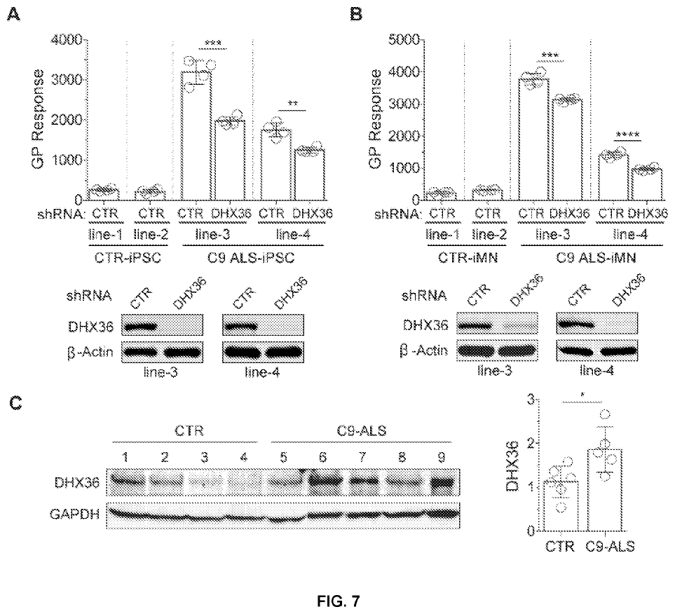

Next, experiments were conducted to test whether DHX36 regulates endogenous DPR protein production in cells from C9orf72-linked ALS patients. An enzyme-linked immunosorbent assay (ELISA) was used that has been optimized for quantification of the levels of poly-GP, one of the five DPRs generated in the patients. The proteins extracted from cells of patients and control individuals, together with biotinylated anti-GP antibodies, were applied to a multi-spot immunoassay platform with high sensitivity (21). As expected, the levels of poly-GP detected in both the C9orf72 ALS patient-derived iPSCs and motor neurons were significantly higher than those in healthy control cells, confirming the sensitivity and specificity of the poly-GP detection assay (FIG. 7A, FIG. 7B). To test the role of DHX36 in the regulation of poly-GP production, DHX36 was knocked down in iPSC lines from C9orf72-linked ALS patients via lentivirus-mediated stable expression of DHX36-specific shRNAs. The data indicated that the poly-GP proteins were significantly decreased in the patient iPSCs upon the DHX36 knockdown when compared to the non-targeting control shRNA treatment (FIG. 7A), indicating that DHX36 is an important positive regulator of endogenous poly-GP production in the cells of patients. Experiments were also conducted to induce differentiation of the iPSCs into motor neurons (iMNs) and test the effects of DHX36 knock-down on poly-GP production. The iMNs were transduced with lentiviruses expressing either DHX36-specific shRNAs or non-targeting control shRNAs for 2 days, followed by culturing for another 8 days before the ELISA analysis. The DHX36 knockdown produced a result similar to that obtained with the iPSCs with a decrease in the levels of poly-GP in the iMNs derived from the C9orf72-linked ALS patients (FIG. 7B), indicating that DHX36 positively regulates RAN translation in patient motor neurons. Furthermore, to determine whether DHX36 is pathologically relevant to C9orf72-linked ALS, expression levels of DHX36 protein were analyzide in spinal cord tissues from five C9orf72-linked ALS patients and six control individuals. The data collected indicated that the average protein levels of DIIX36 in the ALS patient spinal cord tissues were higher than those in the control tissues (FIG. 7C), indicating that DHX36 is abnormally upregulated in the ALS patients.

All publications and patents mentioned in the above specification and indicated in the following References section are herein incorporated by reference in their entirety for all purposes. The following references are cited within this disclosure by referring to the number preceding the reference as indicated below and enclosing the number in parentheses.

Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

### REFERENCES

1. Tran, P. L.; De Cian, A.; Gros, J.; Moriyama, R.; Mergny, J. L., Tetramolecular quadruplex stability and assembly. Topics in current chemistry 2013, 330, 243-73.
2. Yang, D., G-Quadruplex DNA and RNA. Methods Mol Biol 2019, 2035, 1-24.
3. Rhodes, D.; Lipps, H. J., G-quadruplexes and their regu-latory roles in biology. Nucleic Acids Res 2015, 43 (18), 8627-37.
4. Zhang, C.; Liu, H. H.; Zheng, K. W.; Hao, Y. H.; Tan, Z., DNA G-quadruplex formation in response to remote down-stream transcription activity: long-range sensing and signal trans-ducing in DNA double helix. Nucleic Acids Res 2013, 41 (14), 7144-52.
5. Lerner, L. K.; Holzer, S.; Kilkenny, M. L.; Svikovic, S.; Murat, P.; Schiavone, D.; Eldridge, C. B.; Bittleston, A.; Maman, J. D.; Branzei, D.; Stott, K.; Pellegrini, L.; Sale, J. E., Timeless cou-ples G-quadruplex detection with processing by DDX11 helicase during DNA replication. The EMBO journal 2020, 39 (18), e104185.
6. Kharel, P.; Balaratnam, S.; Beals, N.; Basu, S., The role of RNA G-quadruplexes in human diseases and therapeutic strate-gies. Wiley interdisciplinary reviews. RNA 2020, 11 (1), e1568.
7. Spiegel, J.; Adhikari, S.; Balasubramanian, S., The Structure and Function of DNA G-Quadruplexes. Trends Chem 2020, 2 (2), 123-136.
8. Cammas, A.; Millevoi, S., RNA C-quadruplexes: emerg-ing mechanisms in disease. Nucleic Acids Res 2017, 45 (4), 1584-1595.
9. Asamitsu, S.; Obata, S.; Yu, Z.; Bando, T.; Sugiyama, H., Recent Progress of Targeted G-Quadruplex-Preferred Ligands Toward Cancer Therapy. Molecules 2019, 24 (3).
10. Mirkin, S. M., Expandable DNA repeats and human disease. Nature 2007, 447 (7147), 932-40.
11. Orr, H. T.; Zoghbi, H. Y., Trinucleotide repeat disorders. Annu Rev Neurosci 2007, 30, 575-621.
12. La Spada, A. R.; Taylor, J. P., Repeat expansion disease: progress and puzzles in disease pathogenesis. Nature reviews. Ge-netics 2010, 11 (4), 247-58.
13. Todd, P. K.; Paulson, H. L., RNA-mediated neurodegen-eration in repeat expansion disorders. Annals of neurology 2010, 67 (3), 291-300.
14. DeJesus-Hernandez, M.; Mackenzie, I. R.; Boeve, B. F.; Boxer, A. L.; Baker, M.; Rutherford, N. J.; Nicholson, A. M.; Finch, N. A.; Flynn, H.; Adamson, J.; Kouri, N.; Wojtas, A.; Sengdy, P.; Hsiung, G. Y.; Karydas, A.; Seeley, W. W.; Josephs, K. A.; Coppola, G.; Geschwind, D. H.; Wszolek, Z. K.; Feldman, H.; Knopman, D. S.; Petersen, R. C.; Miller, B. L.; Dickson, D. W.; Boylan, K. B.; Graff-Radford, N. R.; Rademakers, R., Expand-ed GGGGCC hexanucleotide repeat in noncoding region of C90RF72 causes chromosome 9p-linked FTD and ALS. Neuron 2011, 72 (2), 245-56.
15. Renton, A. E.; Majounie, E.; Waite, A.; Simon-Sanchez, J.; Rollinson, S.; Gibbs, J. R.; Schymick, J. C.; Laaksovirta, H.; van Swieten, J. C.; Myllykangas, L.; Kalimo, H.; Paetau, A.; Abramzon, Y.; Remes, A. M.; Kaganovich, A.; Scholz, S. W.; Duckworth, J.; Ding, J.; Harmer, D. W.; Hernandez, D. G.; John-son, J. O.; Mok, K.; Ryten, M.; Trabzuni, D.; Guerreiro, R. J.; Orrell, R. W.; Neal, J.; Murray, A.; Pearson, J.; Jansen, I. E.; Sondervan, D.; Seelaar, H.; Blake, D.; Young, K.; Halliwell, N.; Callister, J. B.; Toulson, G.; Richardson, A.; Gerhard, A.; Snow-den, J.; Mann, D.; Neary, D.; Nalls, M. A.; Peuralinna, T.; Jans-son, L.; Isoviita, V. M.; Kaivorinne, A. L.; Holtta-Vuori, M.; Iko-nen, E.; Sulkava, R.; Benatar, M.; Wuu, J.; Chio, A.; Restagno, G.; Borghero, G.; Sabatelli, M.; Consortium, I.; Heckerman, D.; Ro-gaeva, E.; Zinman, L.; Rothstein, J. D.; Sendtner, M.; Drepper, C.; Eichler, E. E.; Alkan, C.; Abdullaev, Z.; Pack, S. D.; Dutra, A.; Pak, E.; Hardy, J.; Singleton, A.; Williams, N. M.; Ileutink, P.; Pickering-Brown, S.; Morris, II. R.; Tienari, P. J.; Traynor, B. J., A hexanucleotide repeat expansion in C90RF72 is the cause of chromosome 9p21 linked ALS-FTD. Neuron 2011, 72 (2), 257-68.
16. Van Langenhove, T.; van der Zee, J.; Van Broeckhoven, C., The molecular basis of the frontotemporal lobar degeneration-amyotrophic lateral sclerosis spectrum. Ann Med 2012, 44 (8), 817-28.
17. Gitler, A. D.; Tsuiji, H., There has been an awakening: Emerging mechanisms of C9orf72 mutations in FTD/ALS. Brain research 2016, 1647, 19-29.
18. Nguyen, L.; Cleary, J. D.; Ranum, L. P. W., Repeat-Associated Non-ATG Translation: Molecular Mechanisms and Contribution to Neurological Disease. Annu Rev Neurosci 2019, 42, 227-247.
19. Goodman, L. D.; Prudencio, M.; Srinivasan, A. R.; Rifai, O. M.; Lee, V. M.; Petrucelli, L.; Bonini, N. M., eIF4B and eIF4H mediate GR production from expanded G4C2 in a Drosophila model for C9orf72-associated ALS. Acta Neuropathol Commun 2019, 7 (1), 62.
20. Yamada, S. B.; Gendron, T. F.; Niccoli, T.; Genuth, N. R.; Grosely, R.; Shi, Y.; Glaria, I.; Kramer, N. J.; Nakayama, L.; Fang, S.; Dinger, T. J. I.; Thoeng, A.; Rocha, G.; Barna, M.; Pu-glisi, J. D.; Partridge, L.; Ichida, J. K.; Isaacs, A. M.; Petrucelli, L.; Gitler, A. D., RPS25 is required for efficient RAN translation of C9orf72 and other neurodegenerative disease-associated nucleotide repeats. Nat Neurosci 2019, 22 (9), 1383-1388.
21. Cheng, W.; Wang, S.; Zhang, Z.; Morgens, D. W.; Hayes, L. R.; Lee, S.; Portz, B.; Xie, Y.; Nguyen, B. V.; Haney, M. S.; Yan, S.; Dong, D.; Coyne, A. N.; Yang, J.; Xian, F.; Cleve-land, D. W.; Qiu, Z.; Rothstein, J. D.; Shorter, J.; Gao, F. B.; Bassik, M. C.; Sun, S., CRISPR-Cas9 Screens Identify the RNA Helicase DDX3X as a Repressor of C90RF72 (GGGGCC)n Re-peat-Associated Non-AUG Translation. Neuron 2019, 104 (5), 885-898 e8.
22. Ciesiolka, A.; Jazurek, M.; Drazkowska, K.; Krzyzosiak, W. J., Structural Characteristics of Simple RNA Repeats Associat-ed with Disease and their Deleterious Protein Interactions. Front Cell Neurosci 2017, 11, 97.
23. Reddy, K.; Zamiri, B.; Stanley, S. Y. Macgregor, R. B., Jr.; Pearson, C. E., The disease-associated r(GGGGCC)n repeat from the C9orf72 gene forms tract length-dependent uni- and mul-timolecular RNA G-quadruplex structures. J Biol Chem 2013, 288 (14), 9860-6.
24. Conlon, E. G.; Lu, L.; Sharma, A.; Yamazaki, T.; Tang, T.; Shneider, N. A.; Manley, J. L., The C90RF72 GGGGCC ex-pansion forms RNA G-quadruplex inclusions and sequesters hnRNP H to disrupt splicing in ALS brains. Elife 2016, 5.
25. Haeusler, A. R.; Donnelly, C. J.; Periz, G.; Simko, E. A.; Shaw, P. G.; Kim, M. S.; Maragakis, N. J.; Troncoso, J. C.; Pan-dey, A.; Sattler, R.; Rothstein, J. D.; Wang, J., C9orf72 nucleotide repeat structures initiate molecular cascades of disease. Nature 2014, 507 (7491), 195-200.
26. Su, Z.; Zhang, Y.; Gendron, T. F.; Bauer, P. O.; Chew, J.; Yang, W. Y.; Fostvedt, E.; Jansen-West, K.; Belzil, V. V.; De-saro, P.; Johnston, A.; Overstreet, K.; Oh, S. Y.; Todd, P. K.; Ber-ry, J. D.; Cudkowicz, M. E.; Boeve, B. F.; Dickson, D.; Floeter, M. K.; Traynor, B. J.; Morelli, C.; Ratti, A.; Silani, V.; Rademak-ers, R.; Brown, R. H.; Rothstein, J. D.; Boylan, K. B.; Petrucelli, L.; Disney, M. D., Discovery of a biomarker and lead small mole-cules to target r(GGGGCC)-associated defects in c9FTD/ALS. Neuron 2014, 83 (5), 1043-50.
27. Chen, M. C.; Tippana, R.; Demeshkina, N. A.; Murat, P.; Balasubramanian, S.; Myong, S.; Ferre-D'Amare, A. R., Structural basis of G-quadruplex unfolding by the DEAH/RHA helicase DHX36. Nature 2018, 558 (7710), 465-469.
28. Lee, H. T.; Sanford, S.; Paul, T.; Choe, J.; Bose, A.; Opresko, P. L.; Myong, S., Position-Dependent Effect of Guanine Base Damage and Mutations on Telomeric G-Quadruplex and Te-lomerase Extension. Biochemistry 2020, 59 (28), 2627-2639.
29. Paul, T.; Voter, A. F.; Cueny, R. R.; Gavrilov, M.; Ha, T.; Keck, J. L.; Myong, S., E. coli Rep helicase and RecA recom-binase unwind G4 DNA and are important for resistance to G4-stabilizing ligands. Nucleic Acids Res 2020, 48 (12), 6640-6653.
30. Roy, R.; Hohng, S.; Ha, T., A practical guide to single-molecule FRET. Nature methods 2008, 5 (6), 507-16.
31. Cheng, W.; Wang, S.; Mestre, A. A.; Fu, C.; Makarem, A.; Xian, F.; Hayes, L. R.; Lopez-Gonzalez, R.; Drenner, K.; Jiang, J.; Cleveland, D. W.; Sun, S., C90RF72 GGGGCC repeat-associated non-AUG translation is upregulated by stress through eIF2alpha phosphorylation. Nature communications 2018, 9 (1), 51.
32. Booy, E. P.; Meier, M.; Okun, N.; Novakowski, S. K.; Xiong, S.; Stetefeld, J.; McKenna, S. A., The RNA helicase RIIAU (DIIX36) unwinds a G4-quadruplex in human telomerase RNA and promotes the formation of the P1 helix template bounda-ry. Nucleic Acids Res 2012, 40 (9), 4110-24.
33. Zu, T.; Liu, Y.; Banez-Coronel, M.; Reid, T.; Pletnikova, O.; Lewis, J.; Miller, T. M.; Harms, M. B.; Falchook, A. E.; Subramony, S. H.; Ostrow, L. W.; Rothstein, J. D.; Troncoso, J. C.; Ranum, L. P., RAN proteins and RNA foci from antisense transcripts in C90RF72 ALS and frontotemporal dementia. Proc Natl Acad Sci USA 2013, 110 (51), E4968-77.
34. Liu, Y.; Wang, T.; Ji, Y. J.; Johnson, K.; Liu, H.; John-son, K.; Bailey, S.; Suk, Y.; Lu, Y. N.; Liu, M.; Wang, J., A C9orf72-CARM1 axis regulates lipid metabolism under glucose starvation-induced nutrient stress. Genes & development 2018, 32 (21-22), 1380-1397.
35. Fratta, P.; Mizielinska, S.; Nicoll, A. J.; Zloh, M.; Fisher, E. M.; Parkinson, G.; Isaacs, A. M., C9orf72 hexanucleotide repeat associated with amyotrophic lateral sclerosis and frontotemporal dementia forms RNA G-quadruplexes. Scientific reports 2012, 2, 1016.
36. Heddi, B.; Cheong, V. V.; Martadinata, H.; Phan, A. T., Insights into G-quadruplex specific recognition by the DEAH-box helicase RHAU: Solution structure of a peptide-quadruplex com-plex. Proc Natl Acad Sci USA 2015, 112 (31), 9608-13.
37. Bhattacharyya, D.; Mirihana Arachchilage, G.; Basu, S., Metal Cations in G-Quadruplex Folding and Stability. Front Chem 2016, 4, 38.
38. Dodd, D. W.; Tomchick, D. R.; Corey, D. R.; Gagnon, K. T., Pathogenic C90RF72 Antisense Repeat RNA Forms a Double Helix with Tandem C:C Mismatches. Biochemistry 2016, 55 (9), 1283-6.
39. Sket, P.; Pohleven, J.; Kovanda, A.; Stalekar, M.; Zu-punski, V.; Zalar, M.; Plavec, J.; Rogelj, B., Characterization of DNA G-quadruplex species forming from C90RF72 G4C2-expanded repeats associated with amyotrophic lateral sclerosis and frontotemporal lobar degeneration. Neurobiology of aging 2015, 36 (2), 1091-6.
40. Adeoye, R. I.; Osalaye, D. S.; Ralebitso, T. K.; Boddis, A.; Reid, A. J.; Fatokun, A. A.; Powell, A. K.; Malomo, S. O.; Olorunniji, F. J., Catalytic Activities of Multimeric G-Quadruplex DNAzymes. Catalysts 2019, 9 (7).
41. Li, W.; Li, Y.; Liu, Z.; Lin, B.; Yi, H.; Xu, F.; Nie, Z.; Yao, S., Insight into G-quadruplex-hemin DNAzyme/RNAzyme: adjacent adenine as the intramolecular species for remarkable en-hancement of enzymatic activity. Nucleic Acids Res 2016, 44 (15), 7373-84.
42. Tippana, R.; Hwang, H.; Opresko, P. L.; Bohr, V. A.; Myong, S., Single-molecule imaging reveals a common mechanism shared by G-quadruplex-resolving helicases. Proc Natl Acad Sci USA 2016, 113 (30), 8448-53.
43. Tippana, R.; Chen, M. C.; Demeshkina, N. A.; Ferre-D'Amare, A. R.; Myong, S., RNA G-quadruplex is resolved by repetitive and ATP-dependent mechanism of DHX36. Nature communications 2019, 10 (1), 1855.
44. Lagier-Tourenne, C.; Baughn, M.; Rigo, F.; Sun, S.; Liu, P.; Li, H. R.; Jiang, J.; Watt, A. T.; Chun, S.; Katz, M.; Qiu, J.; Sun, Y.; Ling, S. C.; Zhu, Q.; Polymenidou, M.; Drenner, K.; Ar-tates, J. W.; McAlonis-Downes, M.; Markmiller, S.; Hutt, K. R.; Pizzo, D. P. Cady, J.; Harms, M. B.; Baloh, R. H.; Vandenberg, S. R.; Yeo, G. W.; Fu, X. D.; Bennett, C. F.; Cleveland, D. W.; Rav-its, J., Targeted degradation of sense and antisense C9orf72 RNA foci as therapy for ALS and frontotemporal degeneration. Proc Natl Acad Sci USA 2013, 110 (47), E4530-9.
45. Endoh, T.; Kawasaki, Y.; Sugimoto, N., Stability of RNA quadruplex in open reading frame determines proteolysis of human estrogen receptor alpha. Nucleic Acids Res 2013, 41 (12), 6222-31.
46. Bourgeois, C. F.; Mortreux, F.; Auboeuf, D., The multi-ple functions of RNA helicases as drivers and regulators of gene expression. Nature reviews. Molecular cell biology 2016, 17 (7), 426-38.
47. Creacy, S. D.; Routh, E. D.; Iwamoto, F.; Nagamine, Y.; Akman, S. A.; Vaughn, J. P., G4 Resolvase 1 Binds Both DNA and RNA Tetramolecular Quadruplex with High Affinity and Is the Major Source of Tetramolecular Quadruplex G4-DNA and G4-RNA Resolving Activity in HeLa Cell Lysates. J Biol Chem 2008, 283 (50), 34626-34634.
48. Liu, H. H.; Zheng, K. W.; He, Y. D.; Chen, Q.; Hao, Y. H.; Tan, Z., RNA G-quadruplex formation in defined sequence in living cells detected by bimolecular fluorescence complementation. Chemical Science 2016, 7 (7), 4573-4581.
49. Guo, J. U.; Bartel, D. P., RNA G-quadruplexes are glob-ally unfolded in eukaryotic cells and depleted in bacteria. Science 2016, 353 (6306).
50. Sauer, M.; Juranek, S. A.; Marks, J.; De Magis, A.; Kazemier, H. G.; Hilbig, D.; Benhalevy, D.; Wang, X.; Hafner, M.; Paeschke, K., DHX36 prevents the accumulation of translationally inactive mRNAs with G4-structures in untranslated regions. Nature communications 2019, 10 (1), 2421.
51. Murat, P.; Marsico, G.; Herdy, B.; Ghanbarian, A.; Por-tella, G.; Balasubramanian, S., RNA G-quadruplexes at upstream open reading frames cause DHX36- and DHX9-dependent transla-tion of human mRNAs. Genome biology 2018, 19 (1), 229.
52. Martone, J.; Mariani, D.; Santini, T.; Setti, A.; Shamloo, S.; Colantoni, A.; Capparelli, F.; Paiardini, A.; Dimartino, D.; Mor-lando, M.; Bozzoni, I., SMaRT lncRNA controls translation of a G-quadruplex-containing mRNA antagonizing the DHX36 helicase. EMBO reports 2020, 21 (6), e49942.
53. Vester, K.; Eravci, M.; Serikawa, T.; Schutze, T.; Weise, C.; Kurreck, J., RNAi-mediated knockdown of the Rhau helicase preferentially depletes proteins with a Guanine-quadruplex motif in the 5′-UTR of their mRNA. Biochemical and biophysical research communications 2019, 508 (3), 756-761.
54. Huppert, J. L.; Balasubramanian, S., Prevalence of quad-ruplexes in the human genome. Nucleic Acids Res 2005, 33 (9), 2908-16.
55. Neidle, S., Quadruplex nucleic acids as targets for anti-cancer therapeutics. Nature Reviews Chemistry 2017, 1 (5), 0041.
56. Ruggiero, E.; Richter, S. N., G-quadruplexes and G-quadruplex ligands: targets and tools in antiviral therapy. Nucleic Acids Res 2018, 46 (7), 3270-3283.
57. Shioda, N.; Yabuki, Y.; Yamaguchi, K.; Onozato, M.; Li, Y.; Kurosawa, K.; Tanabe, H.; Okamoto, N.; Era, T.; Sugiyama, H.; Wada, T.; Fukunaga, K., Targeting G-quadruplex DNA as cognitive function therapy for ATR-X syndrome. Nat Med 2018, 24 (6), 802-813.
58. Butovskaya, E.; Solda, P.; Scalabrin, M.; Nadai, M.; Richter, S. N., HIV-1 Nucleocapsid Protein Unfolds Stable RNA G-Quadruplexes in the Viral Genome and Is Inhibited by G-Quadruplex Ligands. ACS Infect Dis 2019, 5 (12), 2127-2135.
59. Simone, R.; Balendra, R.; Moens, T. G.; Preza, E.; Wil-son, K. M.; Heslegrave, A.; Woodling, N. S.; Niccoli, T.; Gilbert-Jaramillo, J.; Abdelkarim, S.; Clayton, E. L.; Clarke, M.; Konrad, M. T.; Nicoll, A. J.; Mitchell, J. S.; Calvo, A.; Chio, A.; Houlden, H.; Polke, J. M.; Ismail, M. A.; Stephens, C. E.; Vo, T.; Farahat, A. A.; Wilson, W. D.; Boykin, D. W. Zetterberg, H.; Partridge, L.; Wray, S.; Parkinson, G.; Neidle, S.; Patani, R.; Fratta, P. Isaacs, A. M., G-quadruplex-binding small molecules ameliorate C9orf72 FTD/ALS pathology in vitro and in vivo. EMBO Mol Med 2018, 10 (1), 22-31.
60. Wang, Z. F.; Ursu, A.; Childs-Disney, J. L.; Guertler, R.; Yang, W. Y.; Bernat, V.; Rzuczek, S. G.; Fuerst, R.; Zhang, Y. J.; Gendron, T. F.; Yildirim, I.; Dwyer, B. G.; Rice, J. E.; Petrucelli, L.; Disney, M. D., The Hairpin Form of r(G4C2)(exp) in c9ALS/FTD Is Repeat-Associated Non-ATG Translated and a Target for Bioactive Small Molecules. Cell Chem Biol 2019, 26 (2), 179-190 e12.
61. Kumar, V.; Hasan, G. M.; Hassan, M. I., Unraveling the Role of RNA Mediated Toxicity of C9orf72 Repeats in C9-FTD/ALS. Front Neurosci 2017, 11, 711.
62. Zhang, K.; Donnelly, C. J.; Haeusler, A. R.; Crima, J. C.; Machamer, J. B.; Steinwald, P.; Daley, E. L.; Miller, S. J.; Cun-ningham, K. M.; Vidensky, S.; Gupta, S.; Thomas, M. A.; Hong, I.; Chiu, S. L.; Huganir, R. L.; Ostrow, L. W.; Matunis, M. J.; Wang, J.; Sattler, R.; Lloyd, T. E.; Rothstein, J. D., The C9orf72 repeat expansion disrupts nucleocytoplasmic transport. Nature 2015, 525 (7567), 56-61.
63. Fay, M. M.; Anderson, P. J.; Ivanov, P., ALS/FTD-Associated C90RF72 Repeat RNA Promotes Phase Transitions In Vitro and in Cells. Cell reports 2017, 21 (12), 3573-3584.
64. Simko, E. A. J.; Liu, H.; Zhang, T.; Velasquez, A.; Teli, S.; Haeusler, A. R.; Wang, J., G-quadruplexes offer a conserved structural motif for NONO recruitment to NEAT1 architectural lncRNA. Nucleic Acids Res 2020, 48 (13), 7421-7438.
65. Chalupnikova, K.; Lattmann, S.; Selak, N.; Iwamoto, F.; Fujiki, Y.; Nagamine, Y., Recruitment of the RNA helicase RHAU to stress granules via a unique RNA-binding domain. J Biol Chem 2008, 283 (50), 35186-98.
66. Li, Y. R.; King, 0. D.; Shorter, J.; Gitler, A. D., Stress granules as crucibles of ALS pathogenesis. The Journal of cell biology 2013, 201 (3), 361-72.

We claim:

1. A method for treating a patient comprising a C-quadruplex ribonucleic acid (RNA), said method comprising administering to said patient an inhibitor of DHX36.

2. The method of claim 1, wherein said inhibitor of DHX36 is an antibody or antigen-binding antibody fragment.

3. The method of claim 1, wherein said inhibitor of DHX36 is an oligonucleotide or small molecule.

4. The method of claim 1, wherein said patient has a neurodegenerative disease.

5. The method of claim 1, wherein said patient has ALS or FTD.

6. The method of claim 1, further comprising obtaining a sample from the patient and detecting a G-quadruplex RNA in the sample.

7. The method of claim 1, further comprising obtaining a sample from the patient after the administering and detecting a decrease in DHX36 amount or activity in the sample.

8. The method of claim 1, wherein the patient has a hexanucleotide repeat expansion in the C9orf72 gene.

9. A method for treating a patient comprising a hexanucleotide repeat expansion in the C9orf72 gene, said method comprising administering to said patient an inhibitor of DHX36.

10. The method of claim 9, wherein said inhibitor of DHX36 is an antibody or antigen-binding antibody fragment.

11. The method of claim 9, wherein said inhibitor of DHX36 is an oligonucleotide or small molecule.

12. The method of claim 9, wherein said patient has a neurodegenerative disease.

13. The method of claim 9, wherein said patient has ALS or FTD.

14. The method of claim 9, further comprising obtaining a sample from the patient and detecting a hexanucleotide repeat expansion in the C9orf72 gene in the sample.

15. The method of claim 9, further comprising obtaining a sample from the patient after the administering and detecting a decrease in DHX36 amount or activity in the sample.

16. The method of claim 9, wherein the patient comprises a G-quadruplex RNA.

17. A method for treating a patient in need of treatment for a neurodegenerative disease, said method comprising administering to said patient an inhibitor of DHX36.

18. The method of claim 17, wherein said inhibitor of DHX36 is an antibody or antigen-binding antibody fragment.

19. The method of claim 17, wherein said inhibitor of DHX36 is an oligonucleotide or small molecule.

20. The method of claim 17, wherein said neurodegenerative disease is ALS or FTD.

21. The method of claim 17, further comprising obtaining a sample from the patient and detecting a G-quadruplex RNA in the sample.

22. The method of claim 17, further comprising obtaining a sample from the patient and detecting a hexanucleotide repeat expansion in the C9orf72 gene in the sample

23. The method of claim 17, further comprising obtaining a sample from the patient after the administering and detecting a decrease in DHX36 amount or activity in the sample.


SEQUENCE LISTING (28 sequences)

SEQ ID NO: 1 - RNA, 48 nt, synthetic construct
  source 1..48: mol_type = other RNA
ggggccggggccggggccggggccggggccggggccggggccggggcc

SEQ ID NO: 2 - RNA, 24 nt, synthetic construct
  source 1..24: mol_type = other RNA
ggggccggggccggggccggggcc

SEQ ID NO: 3 - RNA, 24 nt, synthetic construct
  source 1..24: mol_type = other RNA
gngnccgngnccgngnccgngncc

SEQ ID NO: 4 - RNA, 24 nt, synthetic construct
  source 1..24: mol_type = other RNA
ccccggccccggccccggccccgg

SEQ ID NO: 5 - DNA, 24 bp, synthetic construct
  source 1..24: mol_type = other DNA
ggggccggggccggggccggggcc

SEQ ID NO: 6 - DNA, 24 bp, synthetic construct
  source 1..24: mol_type = other DNA
ccccggccccggccccggccccgg

SEQ ID NO: 7 - DNA, 24 bp, synthetic construct
  source 1..24: mol_type = other DNA
ccccggccccggccccggccccgg

SEQ ID NO: 8 - DNA, 24 bp, synthetic construct
  source 1..24: mol_type = other DNA
agagaattctggaggtggcggttc

SEQ ID NO: 9 - DNA, 48 bp, synthetic construct
  source 1..48: mol_type = other DNA
agagaattctgaagatgccaaaaacattaagtctggaggtggcggttc

SEQ ID NO: 10 - DNA, 168 bp, synthetic construct
  source 1..168: mol_type = other DNA
ggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtactt

SEQ ID NO: 11 - DNA, 21 bp, synthetic construct
  source 1..21: mol_type = other DNA
gtccgtaactccgatccaaag

SEQ ID NO: 12 - DNA, 21 bp, synthetic construct
  source 1..21: mol_type = other DNA
tgccatagtgcaggatcacct

SEQ ID NO: 13 - DNA, 20 bp, synthetic construct
  source 1..20: mol_type = other DNA
gtgacttcccatttgccacc

SEQ ID NO: 14 - DNA, 20 bp, synthetic construct
  source 1..20: mol_type = other DNA
tgatctggttgccgaagatg

SEQ ID NO: 15 - DNA, 20 bp, synthetic construct
  source 1..20: mol_type = other DNA
gacgtaaacggccacaagtt

SEQ ID NO: 16 - DNA, 20 bp, synthetic construct
  source 1..20: mol_type = other DNA
aagtcgtgctgcttcatgtg

SEQ ID NO: 17 - DNA, 22 bp, synthetic construct
  source 1..22: mol_type = other DNA
gtctcctctgacttcaacagcg

SEQ ID NO: 18 - DNA, 22 bp, synthetic construct
  source 1..22: mol_type = other DNA
accaccctgttgctgtagccaa

SEQ ID NO: 19 - DNA, 22 bp, synthetic construct
  source 1..22: mol_type = other DNA
caccattggcaatgagcggttc

SEQ ID NO: 20 - DNA, 22 bp, synthetic construct
  source 1..22: mol_type = other DNA
aggtctttgcggatgtccacgt

SEQ ID NO: 21 - DNA, 24 bp, synthetic construct
  source 1..24: mol_type = other DNA
accttgctcctgccgagaaagtat

SEQ ID NO: 22 - DNA, 24 bp, synthetic construct
  source 1..24: mol_type = other DNA
atgtttcgcttggtggtcgaatgg

SEQ ID NO: 23 - RNA, 52 nt, synthetic construct
  source 1..52: mol_type = other RNA
nggcgacggcagcgaggcggggccggggccggggccggggccnnnnnnnnnn

SEQ ID NO: 24 - RNA, 18 nt, synthetic construct
  source 1..18: mol_type = other RNA
gccncgcngccgncgcca

SEQ ID NO: 25 - RNA, 6723 nt, Homo sapiens
  source 1..6723: mol_type = unassigned RNA
acagtggcttccgggcccggacgccatttccagcggttgctggttctgacgggttgtagtctgccaggacaatgagttatgactaccatcagaactggggccgtgatgggggtccccgcagctccggtgggggctatggaggggggccagcagggggtcatggaggtaaccgaggctccggaggaggcggcggcggcggagggggtggtcgaggcggcaggggccggcatcccgggcacctgaaaggccgcgaaatcggcatgtggtacgcgaaaaaacaggggcagaagaacaaggaagcggagaggcaagagagagctgtagtacacatggatgaacgacgagaagaacaaattgtacagttactgaattctgttcaagcgaagaatgataaagagtcagaagcacagatatcctggtttgctcctgaggatcatggatacggtactgaagtttctactaagaacacaccatgctcagagaacaaacttgacatccaggaaaagaagttgataaatcaagaaaaaaaaatgtttagaatcaggaacagatcatatattgaccgagattctgagtatctcttgcaagaaaatgaaccagatggaactttagaccaaaaattattggaagatttacaaaagaaaaaaaatgaccttcggtatattgaaatgcagcatttcagagaaaagctgccttcgtatggaatgcaaaaggaattggtaaatttaattgataaccatcaggtaacagtaataagtggtgaaactggttgtggcaaaaccactcaagttactcagttcattttggataactacattgaaagaggaaaaggatctgcttgcagaatagtttgtactcagccaagaagaattagtgccatttcagttgcggaaagagtagctgcagaaagggcagaatcttgtggcagtggtaatagtactggatatcaaattcgtctccagagtcggttgccaaggaaacagggttctatcttatactgtacaacaggaatcatccttcagtggctccagtcagacccgtatttgtccagtgttagtcatatcgtacttgatgaaatccatgaaagaaatctgcagtcagatgttttaatgactgttgttaaagaccttctcaattttcgatctgacttgaaagtaatattgatgagtgcaacattgaatgcagaaaagttttcagaatattttggtaactgtccaatgatacatatacctggttttacctttccggttgtggaatatcttttggaagatgtaattgaaaaaataaggtatgttccagaacaaaaagaacacagatcccagtttaagaggggtttcatgcaagggcatgtaaatagacaagaaaaagaagaaaaagaagcaatatataaagaacgttggccagattatgtaagggaactgcgaagaaggtattctgcaagtactgtagatgttatagaaatgatggaggatgataaagttgatctgaatttgattgttgccctcatccgatacattgttttggaagaagaggatggtgcgatactggtctttctgccaggctgggacaatatcagcactttacatgatctcttgatgtcacaagtaatgtttaaatcagataaatttttaattatacctttacattcactgatgcctacagttaaccagacacaggtgtttaaaagaacccctcctggtgttcggaaaatagtaattgctaccaacattgcggagactagcattaccatagatgatgtcgtttatgtgatagatggaggaaaaataaaagagacgcattttgatactcagaacaatatcagtacaatgtccgctgagtgggttagtaaagctaatgccaaacagagaaaaggtcgagctggaagagttcaacctggtcattgctatcatctgtataatggtcttagagcaagtcttctagatgactatcaactgccagaaattttgagaactcctttggaagaactttgtttacaaataaagattttaaggctaggtggaattgcttattttctgagtagattaatggacccaccatcaaatgaggcagtgttactctccataagacacctgatggagctgaacgctttggataaacaagaagaattgacacctcttggagtccacttggcacgattacccgttgagccacatattggaaaaatgattctttttggagcactgttctgctgcttagacccagtactcactattgctgctagtctcagtttcaaagatccatttgtcattccactgggaaaagaaaagattgcagatgcaagaagaaaggaattggcaaaggatactagaagtgatcacttaacagttgtgaatgcgtttgagggctgggaagaggctaggcgacgtggtttcagatacgaaaaggactattgctgggaatattttctgtcttcaaacacactgcagatgctgcataacatgaaaggacagtttgctgagcatcttcttggagctggatttgtaagcagtagaaatcctaaagatccagaatctaatataaattcagataatgagaagataattaaagctgtcatctgtgctggtttatatcccaaagttgctaaaattcgactaaatttgggtaaaaaaagaaaaatggtaaaagtttacacaaaaaccgatggcctggttgctgttcatcctaaatctgttaatgtggagcaaacagactttcactacaactggcttatctatcacctaaagatgagaacaagcagtatatacttgtatgactgcacagaggtttccccatactgtctcttgttttttggaggtgacatttccatccagaaggataacgatcaggaaactattgctgtagatgagtggattgtatttcagtctccagcaagaattgcccatcttgttaaggaattaagaaaggaactagatattcttctgcaagagaagattgaaagtcctcatcctgtagactggaatgacactaaatccagagactgtgcagtactgtcagctattatagacttgatcaaaacacaggaaaaggcaactcccaggaactttccgccacgattccaggatggatattacagctgacagcttttcaggggtggtctgaaaagccagtttgacagccattcttcatcattgtttaaattttggctggatgccaaaccctgggacatgaacaattttcatgtgtaaggtagaagccttcagtaggtagtaaagacttaatgtgcatgacttgatgttatatgtagagatatatatatatatatatatataccataaaagcaatatgttctctgatcatatactctgctgtggtcatgcccactctttgggagtatattccctttatatatattgagtattgtaccacttgagaaattcctttgttctgttatacaaaattaatctttctgctcataatgattgatgataccaccagtaaaaataggatgtttaccccaaaacaagtgtcaattaagaatttgaacacaaccacattttttaaaatgaaacttctatcggaagtaaattaatttgttgtaataaagtccagtatttaataaaatgtacaatgttaaatctcagccaacctcttggattggtttctacattttcacctatggcttggttcatttgatatttttactatttttaaaacacgtgccctttttatatcaccctgtgcctcataatacagcaattgagagcacggtctggagcctgattacctcagttccagtctcagctctgtcagttagtcagtgtcttggcttctctcctttttccttatcttgtaagatacagttggcccttcatatccatgcattccacatctgtggattcaaacaactgtggatagaagatatttggggaaaaaaaggatggttgtgtctatactgaacatgtgcagacttttttccttgtcattccctaaacaatacagtttaacagctatttatatagtatttacattgtattaggtattataaataatctagagatgacttaaagaatatgggaggttgtgcctaggtaacatgcaaatactacgccattttattataaaagacttgagcatccatggatttaggtatttgaggggttggggttggaaaccagtcccccacagatactgagggacaactgtacatgtaacagttgtgaggatgaagtacattaatacatgtaaagcacataggctagtgcctgtactctctgaatcttagttgttatttgtccctgaaatattttcgtaaatgtctaacaatctattatattacactttttattttattgctacttatcttcttcgccaaggaatagtgtaacaacacatgctgtacattttcagccctatcagattgtgataaatactgttggttgccggccagcaactattttgtccctcccttcttgcttgcttattctggtgttcccttctgtgcagctctgtgtccaggtatgttggaccctttccctaagggtgaatcctgctgttctaaatgaatcacatgtggtcttatcccccttataagtaattgataaggcatgagtacataaacatgtcctagtgaatggcagctgagaagtgatcttcaagggggtgtctgaaaaatgttttactcactaataggcttagaggaggaaatcctgctctctctggacattgtcttttctgactctgatagttggaactgctgcagccaactaatgatcacaaggagacacggactcagactgaggttagctcagtggaaagatagaactcaccagagcctttgataatgccggtaagcttcagaattaacccattctagagctgctttacatgtggacttatgttgatataataaaaacccttcaattgtttaagccacttttaataggatattgttagttttttgttggttttttgttttgttttgttttgttttttgttttttggagacagtgtcttgccctgtcacccaggctggagtgcagtggtgtgatcttggttcactgcagcctcgacttcctgggttcaagtgatccttctgcctcagcctcccaaatagctgggactataggcatgcatcaccacacccagctaatttctgtattttttgtagagacaccgtttcaccatgttgcccaggctggtctcaaaactcctgtgctcaagcagtctgcccgccttggcttcccaaagtgctgacattacaggtatgagccactgcacccggtccctagtagggtatttttaacttggcaaaaagggtattaatcagaaacatacactatccatcactgagtattatactcttgattttgcaatgtaagagttttaacctatttgtctgacagaaggtgtaaaagtgctggacatgctgcagaaagaaacaggaaaagcatcttgctgcactccaaagaatgcaaattttaatgtgccttccaataaatgcaaggaaaccgaaagagttaatagtatcatacaatgactgggatgtattgataatgctacagtgatccattaacagcacttgttcaggtactgctcccaatcctgggaatgtgtgttacctgttatgtctcaaatcctttatcttggctgttcttctaaaagtgatctgtctgtcattgaatgctggcattagagtcatgtcacaatattagagcatactggttttgcctctaacgtcgaggtgaccatctggtctttcttgctctgtaagagtttctgtttgtgcttttttttcccttacttttattatccagtttaaatccagtcagttggccaaaatggtcttggaagaaaggatttagagattctgtaaaaaagattattagttggttctgtgtttgcaaaccagtatgtaattcccatatgaatggggaaaatgttcctgaggaattatttaattgatgaggcattcacagagattaaagcagttattgttacctccaatttatgtcttctgcacagctgattaggttggacattttaatccaaggtgctacttacattctgtttcttttgtttacaaaatagggcattactgagtagaaaatacattccaggtctaagaactgtttgttctttagtggattttaaagaataagacattaaattaagaaggaaaatctgtttgattcatcattttgagtaatgcagagaataactcttaaaaaaaaaacctgaaacagaaactttcaggaaaaaaacaaaagaaattaaactactgaataatctttcaaaagcatcgttgaacagtgagcacatgtgatgccttcccaattaattaatttgggcaaaagctctagataaacttacaccaattgttgcaaggcattctctattactgcatttttaaaacaaagtcctcccctaaaagttatgaattatacaaaagctttaggaggaattttagtactcctttacccaaaataaatagtggtaaatttccttctgttcttagctctgagtttgtatttatttataagtcattgtgatcatgtatacttatatatttttaatatgctaattgcttttaaatgggatttttgttctgattttaaaataatacatcctccctggacaatatttggaaatgtagcaaagtaaatcatgttaccattgttccattttgatatatataaactttaggtattattttatatataataaattttaagatattatatacaataaaattaaccaatttaa

SEQ ID NO: 26 - RNA, 6681 nt, Homo sapiens
  source 1..6681: mol_type = unassigned RNA
acagtggcttccgggcccggacgccatttccagcggttgctggttctgacgggttgtagtctgccaggacaatgagttatgactaccatcagaactggggccgtgatgggggtccccgcagctccggtgggggctatggaggggggccagcagggggtcatggaggtaaccgaggctccggaggaggcggcggcggcggagggggtggtcgaggcggcaggggccggcatcccgggcacctgaaaggccgcgaaatcggcatgtggtacgcgaaaaaacaggggcagaagaacaaggaagcggagaggcaagagagagctgtagtacacatggatgaacgacgagaagaacaaattgtacagttactgaattctgttcaagcgaagaatgataaagagtcagaagcacagatatcctggtttgctcctgaggatcatggatacggtactgaagtttctactaagaacacaccatgctcagagaacaaacttgacatccaggaaaagaagttgataaatcaagaaaaaaaaatgtttagaatcaggaacagatcatatattgaccgagattctgagtatctcttgcaagaaaatgaaccagatggaactttagaccaaaaattattggaagatttacaaaagaaaaaaaatgaccttcggtatattgaaatgcagcatttcagagaaaagctgccttcgtatggaatgcaaaaggaattggtaaatttaattgataaccatcaggtaacagtaataagtggtgaaactggttgtggcaaaaccactcaagttactcagttcattttggataactacattgaaagaggaaaaggatctgcttgcagaatagtttgtactcagccaagaagaattagtgccatttcagttgcggaaagagtagctgcagaaagggcagaatcttgtggcagtggtaatagtactggatatcaaattcgtctccagagtcggttgccaaggaaacagggttctatcttatactgtacaacaggaatcatccttcagtggctccagtcagacccgtatttgtccagtgttagtcatatcgtacttgatgaaatccatgaaagaaatctgcagtcagatgttttaatgactgttgttaaagaccttctcaattttcgatctgacttgaaagtaatattgatgagtgcaacattgaatgcagaaaagttttcagaatattttggtaactgtccaatgatacatatacctggttttacctttccggttgtggaatatcttttggaagatgtaattgaaaaaataaggtatgttccagaacaaaaagaacacagatcccagtttaagaggggtttcatgcaagggcatgtaaatagacaagaaaaagaagaaaaagaagcaatatataaagaacgttggccagattatgtaagggaactgcgaagaaggtattctgcaagtactgtagatgttatagaaatgatggaggatgataaagttgatctgaatttgattgttgccctcatccgatacattgttttggaagaagaggatggtgcgatactggtctttctgccaggctgggacaatatcagcactttacatgatctcttgatgtcacaagtaatgtttaaatcagttaaccagacacaggtgtttaaaagaacccctcctggtgttcggaaaatagtaattgctaccaacattgcggagactagcattaccatagatgatgtcgtttatgtgatagatggaggaaaaataaaagagacgcattttgatactcagaacaatatcagtacaatgtccgctgagtgggttagtaaagctaatgccaaacagagaaaaggtcgagctggaagagttcaacctggtcattgctatcatctgtataatggtcttagagcaagtcttctagatgactatcaactgccagaaattttgagaactcctttggaagaactttgtttacaaataaagattttaaggctaggtggaattgcttattttctgagtagattaatggacccaccatcaaatgaggcagtgttactctccataagacacctgatggagctgaacgctttggataaacaagaagaattgacacctcttggagtccacttggcacgattacccgttgagccacatattggaaaaatgattctttttggagcactgttctgctgcttagacccagtactcactattgctgctagtctcagtttcaaagatccatttgtcattccactgggaaaagaaaagattgcagatgcaagaagaaaggaattggcaaaggatactagaagtgatcacttaacagttgtgaatgcgtttgagggctgggaagaggctaggcgacgtggtttcagatacgaaaaggactattgctgggaatattttctgtcttcaaacacactgcagatgctgcataacatgaaaggacagtttgctgagcatcttcttggagctggatttgtaagcagtagaaatcctaaagatccagaatctaatataaattcagataatgagaagataattaaagctgtcatctgtgctggtttatatcccaaagttgctaaaattcgactaaatttgggtaaaaaaagaaaaatggtaaaagtttacacaaaaaccgatggcctggttgctgttcatcctaaatctgttaatgtggagcaaacagactttcactacaactggcttatctatcacctaaagatgagaacaagcagtatatacttgtatgactgcacagaggtttccccatactgtctcttgttttttggaggtgacatttccatccagaaggataacgatcaggaaactattgctgtagatgagtggattgtatttcagtctccagcaagaattgcccatcttgttaaggaattaagaaaggaactagatattcttctgcaagagaagattgaaagtcctcatcctgtagactggaatgacactaaatccagagactgtgcagtactgtcagctattatagacttgatcaaaacacaggaaaaggcaactcccaggaactttccgccacgattccaggatggatattacagctgacagcttttcaggggtggtctgaaaagccagtttgacagccattcttcatcattgtttaaattttggctggatgccaaaccctgggacatgaacaattttcatgtgtaaggtagaagccttcagtaggtagtaaagacttaatgtgcatgacttgatgttatatgtagagatatatatatatatatatatataccataaaagcaatatgttctctgatcatatactctgctgtggtcatgcccactctttgggagtatattccctttatatatattgagtattgtaccacttgagaaattcctttgttctgttatacaaaattaatctttctgctcataatgattgatgataccaccagtaaaaataggatgtttaccccaaaacaagtgtcaattaagaatttgaacacaaccacattttttaaaatgaaacttctatcggaagtaaattaatttgttgtaataaagtccagtatttaataaaatgtacaatgttaaatctcagccaacctcttggattggtttctacattttcacctatggcttggttcatttgatatttttactatttttaaaacacgtgccctttttatatcaccctgtgcctcataatacagcaattgagagcacggtctggagcctgattacctcagttccagtctcagctctgtcagttagtcagtgtcttggcttctctcctttttccttatcttgtaagatacagttggcccttcatatccatgcattccacatctgtggattcaaacaactgtggatagaagatatttggggaaaaaaaggatggttgtgtctatactgaacatgtgcagacttttttccttgtcattccctaaacaatacagtttaacagctatttatatagtatttacattgtattaggtattataaataatctagagatgacttaaagaatatgggaggttgtgcctaggtaacatgcaaatactacgccattttattataaaagacttgagcatccatggatttaggtatttgaggggttggggttggaaaccagtcccccacagatactgagggacaactgtacatgtaacagttgtgaggatgaagtacattaatacatgtaaagcacataggctagtgcctgtactctctgaatcttagttgttatttgtccctgaaatattttcgtaaatgtctaacaatctattatattacactttttattttattgctacttatcttcttcgccaaggaatagtgtaacaacacatgctgtacattttcagccctatcagattgtgataaatactgttggttgccggccagcaactattttgtccctcccttcttgcttgcttattctggtgttcccttctgtgcagctctgtgtccaggtatgttggaccctttccctaagggtgaatcctgctgttctaaatgaatcacatgtggtcttatcccccttataagtaattgataaggcatgagtacataaacatgtcctagtgaatggcagctgagaagtgatcttcaagggggtgtctgaaaaatgttttactcactaataggcttagaggaggaaatcctgctctctctggacattgtcttttctgactctgatagttggaactgctgcagccaactaatgatcacaaggagacacggactcagactgaggttagctcagtggaaagatagaactcaccagagcctttgataatgccggtaagcttcagaattaacccattctagagctgctttacatgtggacttatgttgatataataaaaacccttcaattgtttaagccacttttaataggatattgttagttttttgttggttttttgttttgttttgttttgttttttgttttttggagacagtgtcttgccctgtcacccaggctggagtgcagtggtgtgatcttggttcactgcagcctcgacttcctgggttcaagtgatccttctgcctcagcctcccaaatagctgggactataggcatgcatcaccacacccagctaatttctgtattttttgtagagacaccgtttcaccatgttgcccaggctggtctcaaaactcctgtgctcaagcagtctgcccgccttggcttcccaaagtgctgacattacaggtatgagccactgcacccggtccctagtagggtatttttaacttggcaaaaagggtattaatcagaaacatacactatccatcactgagtattatactcttgattttgcaatgtaagagttttaacctatttgtctgacagaaggtgtaaaagtgctggacatgctgcagaaagaaacaggaaaagcatcttgctgcactccaaagaatgcaaattttaatgtgccttccaataaatgcaaggaaaccgaaagagttaatagtatcatacaatgactgggatgtattgataatgctacagtgatccattaacagcacttgttcaggtactgctcccaatcctgggaatgtgtgttacctgttatgtctcaaatcctttatcttggctgttcttctaaaagtgatctgtctgtcattgaatgctggcattagagtcatgtcacaatattagagcatactggttttgcctctaacgtcgaggtgaccatctggtctttcttgctctgtaagagtttctgtttgtgcttttttttcccttacttttattatccagtttaaatccagtcagttggccaaaatggtcttggaagaaaggatttagagattctgtaaaaaagattattagttggttctgtgtttgcaaaccagtatgtaattcccatatgaatggggaaaatgttcctgaggaattatttaattgatgaggcattcacagagattaaagcagttattgttacctccaatttatgtcttctgcacagctgattaggttggacattttaatccaaggtgctacttacattctgtttcttttgtttacaaaatagggcattactgagtagaaaatacattccaggtctaagaactgtttgttctttagtggattttaaagaataagacattaaattaagaaggaaaatctgtttgattcatcattttgagtaatgcagagaataactcttaaaaaaaaaacctgaaacagaaactttcaggaaaaaaacaaaagaaattaaactactgaataatctttcaaaagcatcgttgaacagtgagcacatgtgatgccttcccaattaattaatttgggcaaaagctctagataaacttacaccaattgttgcaaggcattctctattactgcatttttaaaacaaagtcctcccctaaaagttatgaattatacaaaagctttaggaggaattttagtactcctttacccaaaataaatagtggtaaatttccttctgttcttagctctgagtttgtatttatttataagtcattgtgatcatgtatacttatatatttttaatatgctaattgcttttaaatgggatttttgttctgattttaaaataatacatcctccctggacaatatttggaaatgtagcaaagtaaatcatgttaccattgttccattttgatatatataaactttaggtattattttatatataataaattttaagatattatatacaataaaattaaccaatttaa

SEQ ID NO: 27 - protein, 1008 aa, Homo sapiens
MSYDYHQNWGRDGGPRSSGGGYGGGPAGGHGGNRGSGGGGGGGGGGRGGRGRHPGHLKGREIGMWYAKKQGQKNKEAERQERAVVHMDERREEQIVQLLNSVQAKNDKESEAQISWFAPEDHGYGTEVSTKNTPCSENKLDIQEKKLINQEKKMFRIRNRSYIDRDSEYLLQENEPDGTLDQKLLEDLQKKKNDLRYIEMQHFREKLPSYGMQKELVNLIDNHQVTVISGETGCGKTTQVTQFILDNYIERGKGSACRIVCTQPRRISAISVAERVAAERAESCGSGNSTGYQIRLQSRLPRKQGSILYCTTGIILQWLQSDPYLSSVSHIVLDEIHERNLQSDVLMTVVKDLLNFRSDLKVILMSATLNAEKFSEYFGNCPMIHIPGFTFPVVEYLLEDVIEKIRYVPEQKEHRSQFKRGFMQGHVNRQEKEEKEAIYKERWPDYVRELRRRYSASTVDVIEMMEDDKVDLNLIVALIRYIVLEEEDGAILVFLPGWDNISTLHDLLMSQVMFKSDKFLIIPLHSLMPTVNQTQVFKRTPPGVRKIVIATNIAETSITIDDVVYVIDGGKIKETHFDTQNNISTMSAEWVSKANAKQRKGRAGRVQPGHCYHLYNGLRASLLDDYQLPEILRTPLEELCLQIKILRLGGIAYFLSRLMDPPSNEAVLLSIRHLMELNALDKQEELTPLGVHLARLPVEPHIGKMILFGALFCCLDPVLTIAASLSFKDPFVIPLGKEKIADARRKELAKDTRSDHLTVVNAFEGWEEARRRGFRYEKDYCWEYFLSSNTLQMLHNMKGQFAEHLLGAGFVSSRNPKDPESNINSDNEKIIKAVICAGLYPKVAKIRLNLGKKRKMVKVYTKTDGLVAVHPKSVNVEQTDFHYNWLIYHLKMRTSSIYLYDCTEVSPYCLLFFGGDISIQKDNDQETIAVDEWIVFQSPARIAHLVKELRKELDILLQEKIESPHPVDWNDTKSRDCAVLSAIIDLIKTQEKATPRNFPPRFQDGYYS

SEQ ID NO: 28 - protein, 994 aa, Homo sapiens
MSYDYHQNWGRDGGPRSSGGGYGGGPAGGHGGNRGSGGGGGGGGGGRGGRGRHPGHLKGREIGMWYAKKQGQKNKEAERQERAVVHMDERREEQIVQLLNSVQAKNDKESEAQISWFAPEDHGYGTEVSTKNTPCSENKLDIQEKKLINQEKKMFRIRNRSYIDRDSEYLLQENEPDGTLDQKLLEDLQKKKNDLRYIEMQHFREKLPSYGMQKELVNLIDNHQVTVISGETGCGKTTQVTQFILDNYIERGKGSACRIVCTQPRRISAISVAERVAAERAESCGSGNSTGYQIRLQSRLPRKQGSILYCTTGIILQWLQSDPYLSSVSHIVLDEIHERNLQSDVLMTVVKDLLNFRSDLKVILMSATLNAEKFSEYFGNCPMIHIPGFTFPVVEYLLEDVIEKIRYVPEQKEHRSQFKRGFMQGHVNRQEKEEKEAIYKERWPDYVRELRRRYSASTVDVIEMMEDDKVDLNLIVALIRYIVLEEEDGAILVFLPGWDNISTLHDLLMSQVMFKSVNQTQVFKRTPPGVRKIVIATNIAETSITIDDVVYVIDGGKIKETHFDTQNNISTMSAEWVSKANAKQRKGRAGRVQPGHCYHLYNGLRASLLDDYQLPEILRTPLEELCLQIKILRLGGIAYFLSRLMDPPSNEAVLLSIRHLMELNALDKQEELTPLGVHLARLPVEPHIGKMILFGALFCCLDPVLTIAASLSFKDPFVIPLGKEKIADARRKELAKDTRSDHLTVVNAFEGWEEARRRGFRYEKDYCWEYFLSSNTLQMLHNMKGQFAEHLLGAGFVSSRNPKDPESNINSDNEKIIKAVICAGLYPKVAKIRLNLGKKRKMVKVYTKTDGLVAVHPKSVNVEQTDFHYNWLIYHLKMRTSSIYLYDCTEVSPYCLLFFGGDISIQKDNDQETIAVDEWIVFQSPARIAHLVKELRKELDILLQEKIESPHPVDWNDTKSRDCAVLSAIIDLIKTQEKATPRNFPPRFQDGYYS